(19) United States
(12) Patent Application Publication
(10) Pub. No.: US 2018/0000465 A1
(43) Pub. Date: Jan. 4, 2018

(54) INTEGRATED PAINLESS BONE MARROW BIOPSY DEVICE

(71) Applicant: YALE UNIVERSITY, NEW HAVEN, CT (US)

(72) Inventors: Elliott BROWN, New Haven, CT (US); Tamir FRIEDMAN, New Haven, CT (US)

(21) Appl. No.: 15/548,197

(22) PCT Filed: Jan. 12, 2016
(86) PCT No.: PCT/IB2016/050123

§ 371 Date: Aug. 2, 2017

Related U.S. Application Data
(60) Provisional application No. 62/110,699, filed on Feb. 2, 2015.

(51) Int. Cl. A61B 10/02; A61M 19/00; A61B 17/34; A61M 5/32

(52) U.S. Cl. CPC A61B 10/025; A61M 5/3291; A61M 19/00; A61B 17/3403; A61B 2010/0258; A61B 2017/3413; A61B 2217/005

(57) ABSTRACT

An apparatus and method for performing painless bone marrow biopsy that requires only a single procedural pass to the biopsy bone site is disclosed. The biopsy device combines dermatotomy, anesthesia and marrow specimen extraction functionality and inherently aligns the bone drilling site with the site of anesthetic delivery by incorporating a transport channel that may be used for anesthetic delivery, into the biopsy needle used for bone access and bone marrow sample retrieval. The biopsy device also discloses a right angle drill interface and multiple side-access ports for aspiration and anesthetic syringe attachments. A novel syringe/plunger system comprising vacuum tube fitted plungers for quick transfer of marrow specimen sample without requiring a second non-sterile assistant is also disclosed.

### FIELD OF THE INVENTION

The present invention relates to a method and device for tissue extraction and instrumentation.

### RELATED ART

Components of blood are made in the bone marrow. As illustrated in FIG. 1, bone marrow generally refers to the flexible tissue or spongy material contained within the central bony compartment of some larger bones. Bone marrow biopsy and bone marrow aspiration related to techniques and procedures for collecting a small sample of the bone marrow inside the bones for subsequent testing in a laboratory. These procedures are used to diagnose and monitor blood and marrow disease, such as anemia and hematopoietic (blood) malignancies. Bone marrow has a fluid portion and a more solid portion. In a bone marrow biopsy, a biopsy needle is used to withdraw a sample of the solid portion. In bone marrow aspiration, a needle is used to withdraw a sample of the fluid portion. Bone marrow analysis often involves performing a bone marrow biopsy and bone marrow aspiration at the same time. Analysis may generally consist of three components, namely, cytogenetic, flow cytometry and trabecular bone core biopsy. Nearly 100,000 bone marrow biopsies are performed each year according to the Medicaid registry alone; these only account for hospital based procedures and do not take into account the overwhelming majority of biopsies performed in the outpatient office setting. The procedure is reliably painful and most commonly done without image guidance, resulting in both dangers to the patient, as well as discomfort. Performing the procedure with CT guidance and general anesthesia may lessen the discomfort experienced by the patient, but it adds significant cost to the procedure and it involves exposure to radiation for both patient and the clinician.

### SUMMARY

According to one broad aspect, the present invention provides a device comprising: a needle assembly comprising: a hollow tubular sleeve having a hollow opening therethrough, a stylet mounted in the hollow opening of the hollow tubular sleeve and having a first conduit therethrough, and one or more side openings, a liquid pressure generator in fluid communication with the first conduit and configured to apply pressure to liquid in the first conduit, wherein when a container containing a liquid anesthetic agent is in fluid communication with the first conduit and the liquid pressure generator applies pressure to the liquid anesthetic agent, the liquid anesthetic agent is forced through the first conduit and out through one or more end-openings of the stylet, and wherein when the device is inserted in bone marrow so that side openings are exposed to the bone marrow and a suction generator applies suction to a second conduit extending at least partway through the needle assembly, at least a portion of the bone marrow is withdrawn into the device through the side openings.

According to a second broad aspect, the present invention provides a method comprising administering a liquid anesthetic to a site of drilling in a bone through an opening in a tip of a needle assembly drilling into the bone as the needle assembly drills into the bone at the site of drilling.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a diagram showing the structure of a human femur (thigh bone) including a compact cortex on top of spongy bone layer containing the marrow and the blood cells that are produced by and replenished from the marrow.

FIG. 2 is a diagram showing a conventional biopsy needle and bone access process through the soft tissue and the compact cortex layer into the region holding the bone marrow.

FIG. 3 is an image of a conventional spinal needle used for local anesthesia prior to drilling into bone.

FIG. 4 is a schematic illustration of an integrated biopsy/aspiration needle device used to consolidate anesthesia and marrow access/extraction into a one pass needle trip to the bone according to one embodiment of the present invention.

FIG. 5 is a schematic illustration of the integrated biopsy/aspiration needle device of FIG. 4 in which a needle of the device is shown separated from cannula in which the needle is inserted.

FIG. 6 is a side view of an anesthetic agent delivery needle and skin cutting blade combination device according to one embodiment of the present invention.

FIG. 7 is a schematic illustration of a biopsy needle with a central anesthetic agent channel comprising multiple holes for anesthetic agent dispersal through the side and the bottom sections of the channel according to one embodiment of the present invention.

FIG. 8 is an enlarged view of a region labeled “A” in FIG. 7.

FIG. 9 is schematic illustration a broader application of the biopsy needle of FIG. 7 wherein the central anesthetic channel is further used for application of suction to facilitate retrieval of tissue sample, according to one embodiment of the present invention.

FIG. 10 is an enlarged view of a region labeled “B” in FIG. 9.

FIG.11 is schematic illustration of additional features of the biopsy needle of FIG. 7, where the outer sleeve is “fired” over the top of the biopsy trough to cut a specimen, with or without the addition of suction as illustrated in FIG. 9, according to one embodiment of the present invention.

FIG. 12 is an enlarged view of a region labeled “C” in FIG. 11.

FIG. 13. is a side view of an integrated one-pass biopsy needle assembly with a slip joint and a right angle gear configuration, according to one embodiment of the present invention.

FIG. 14 is a side view of a configuration of a biopsy needle assembly with two aspiration syringes connected thereto and the inner stylet withdrawn, according to one embodiment of the present inversion.

FIG. 15 is a side view of a biopsy needle assembly with anesthetic syringe loaded onto the needle body and connected to an anesthetic delivery channel integrated within the needle, which may exist with or without the aspiration syringes illustrated in FIG. 14, according to one embodiment of the present invention.

FIG. 16 is a diagram showing a needle drill bit that is optimized for drilling bone, with novel cutting edges on the distal end and a novel configuration of side and end holes in order facilitate bone surface and intraosseous anesthetic infusion, according to one embodiment of the present invention.

FIG. 17 is an enlarged view of the end of the needle drill bit of FIG. 16.

FIG. 18 is a schematic illustration of a syringe/plunger system of syringe plungers fitted with vacuum tubes and a rubber-covered needle that spans the rubber part of the plunger into the syringe according to one embodiment of the present invention.

FIG. 19 a schematic illustration of the parts of the syringe/plunger system of FIG. 18 in unassembled form.

FIG. 20 is a diagram showing a syringe/plunger system for direct and quick transfer of bone marrow specimen into vacuum tubes without risking contamination from a second non-sterile assisting person according to one embodiment of the present invention.

FIG. 21 is a side view of an integrated one-pass biopsy needle assembly having a drill interface, side-access ports with stationary cover for multiple attachments of anesthetic and/or aspiration syringes and a distal drill bit optimized for bone drilling according to one embodiment of the present invention.

FIG. 22 is a diagram illustration of an ultrasound-guided integrated one-pass biopsy device equipped with laser guidance for marking the skin entry site according to one embodiment of the present invention.

FIG. 23 is an image of an ultrasound display capture upon initial placement of the ultrasound probe.

FIG. 24 is an image of a biopsy needle guide adjustment procedure used to plan correct, perpendicular contact with the biopsy target bone site using the ultrasound display.

FIG. 25 is an image of an ultrasound display capture showing a correct planned needle-bone angle.

FIG. 26 is an image of an ultrasound beam angle adjustment procedure for optimum needle visualization.

FIG. 27 is an image of an ultrasound display capture showing a correct ultrasound beam angle for optimum needle visualization.

FIG. 28 is an image showing a real-time adjustment of a probe for maintaining optimal needle visualization as the needle is advanced towards a target bone surface.

FIG. 29 is an image of an ultrasound display capture showing an ultrasound-guided biopsy needle delivering anesthetic into soft tissue for anesthetizing the path of the biopsy needle as it advances towards a target biopsy bone site.

FIG. 30 is an image of a periosteum lidocaine delivery once an ultra-sound guided biopsy needle contacts a bone surface.

FIG. 31 illustrates an intraosseous delivery of lidocaine through end and side holes at a distal tip of a biopsy needle once a hard cortex at the biopsy target site is breached and the biopsy needle is advanced into the marrow cavity space.

FIG. 32 is an image showing a retraction of a central stylet from a biopsy needle in preparation for a bone marrow aspiration.

FIG. 33 is an image showing multiple simultaneous bone marrow aspirations through a biopsy needle.

FIG. 34 is an image showing a transfer of collected bone marrow aspirate into vacuum tubes fitted within a plunger of an aspiration syringe.

FIG. 35 is an image showing a process of retrieving solid marrow core sample by advancing a rotating biopsy needle into a marrow cavity and withdrawing the needle after sufficient amount of core specimen has been pushed into a channel cavity formed by the retraction of a central stylet from the biopsy needle.

FIG. 36 is a cross-sectional view of an end of a biopsy needle.

FIG. 37 is a distal end of a needle assembly according to one embodiment of the present invention.

FIG. 38 is a cross-sectional view of the distal end of the needle assembly of FIG. 37.

FIG. 39 is a perspective view of a drill device according to one embodiment of the present invention.

FIG. 40 is perspective view of the drill device of FIG. 39 in which some parts of the drill device are shown as translucent to allow interior parts of the drill device to be visible.

FIG. 41 is a perspective view of the drill device of FIG. 39 with parts of the drill device removed to show interior structures.

FIG. 42 is a perspective view of the drill device of FIG. 39 with some parts of the drill device removed and some parts of the drill device shown as translucent to make interior structures visible.

FIG. 43 is a side view of the drill device of FIG. 39 in which some parts of the drill device are shown as translucent to allow interior parts of the drill device to be visible.

FIG. 44 is a perspective view of part of the drill device of FIG. 43 with part of the drill device shown as translucent to allow interior parts of the drill device to be visible.

FIG. 45 is perspective view of a portion of the part of the drill device shown in FIG. 44 with a part of the drill device removed to shown interior structures.

FIG. 46 is a drawing in schematic form showing the control apparatus for a drill apparatus according to one embodiment of the present invention.

FIG. 47 is a side view of an ultrasound guidance device according to one embodiment of the present invention.

FIG. 48 is a top exploded view of the ultrasound guidance device of FIG. 47.

FIG. 49 is a top view of the interface between two components of the ultrasound guidance device of FIG. 47.

FIG. 50 is a perspective view of the interface between two components of the ultrasound guidance device of FIG. 47.

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

For purposes of the present invention, it should be noted that the singular forms, “a,” “an” and “the,” include reference to the plural unless the context as herein presented clearly indicates otherwise.

For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, a value or property is “based” on a particular value, property, the satisfaction of a condition or other factor if that value is derived by performing a mathematical calculation or logical operation using that value, property or other factor.

For purposes of the present invention, the term “anesthetic” and the term “anesthetic agent” refer to an agent that produces a reversible loss of sensation in an area of a subject's body. An example of an anesthetic is lidocaine. An anesthetic may be in gaseous form, liquid form, etc. An anesthetic in liquid form may a single liquid, a solution, an emulsion, etc.

For purposes of the present invention, the term “anticoagulant” refers to a substance that delays or prevents the formation of blood clots. An example of anticoagulant is heparin.

For purposes of the present invention, the term “axially integrated” refers to a condition being integrated along the longitudinal axis of a structure.

For purposes of the present invention, the term “bone-access needle” refers to a device used to access the bone marrow cavity space through the hard cortex of a bone.

For purposes of the present invention, the term “distal” refers to the bottom end of a device remote from point of attachment or origin. In disclosed embodiment, distal refers to the end furthest away from a medical professional when introducing a device in a patient.

For purposes of the present invention, the term “infusion” refers to a process of slow introduction of an element, for example a solution, into or onto a target.

For purposes of the present invention, the term “intramedullary space” refers to the space within the marrow cavity of a bone.

For purposes of the present invention, the term “intraosseous infusion” refers to the process of injecting a therapeutic agent directly into the marrow of a bone.

For purposes of the present invention, the term “fluid communication” refers to fluid, such as a gas or liquid, being able to flow from one device to another device through one or more channels, conduits, tubes, etc. For example, in one embodiment of the present invention a container containing a liquid anesthetic agent may be in liquid communication with a conduit extending through a central stylet thereby allowing the liquid anesthetic agent to flow from the container containing the liquid anesthetic agent into the conduit of the central stylet through one or more tubes, catheters, cannulas, etc. connecting the container containing the liquid anesthetic agent to the conduit of the central stylet. The container may be a syringe, pouch, plastic bag, bottle, etc., or any other suitable type of container.

For purposes of the present invention, the term “lumen” refers to a canal, duct or cavity within a tubular structure.

For purposes of the present invention, the term “liquid pressure generator” refers to any type of device that can be used to force a liquid through one or more tubes, conduits, channels, etc. Examples of pressure generators include a syringe, an air pump, a rubber bulb, etc.

For purposes of the present invention, the term “proximal” refers to the closest end of a device situated nearer to the center of the body or the point of attachment. In disclosed embodiments, proximal refers to the end closest to a medical professional when placing a device in the patient.

For purposes of the present invention, the term “real-time” refers to a live or low latency processing of incoming data. Events are depicted as occurring entirely within the span of and at the same rate as the depiction.

For purposes of the present invention, the term “site of bone entry” site where a tip of a drill, such as a needle, enters a bone.

For purposes of the present invention, the term “site of drilling” with respect to a bone refers to the region through which a tip of a drill, such as a needle, is enters bone.

For purposes of the present invention, the term “suction generator” refers to any type of devices that generates suction. Examples of suction generators include a syringe, a rubber bulb, a suction pump, etc.

For purposes of the present invention, the term “transport channel” refers to a conduit, duct or any type of longitudinal hollow path-way used for transport in either longitudinal direction. For example, a transport channel maybe used for the delivery of an anesthetic agent down the transport channel from a syringe to target anatomical site or a transport channel maybe used for the transport of tissue or cell samples up the transport channel from an anatomical site or a lesion into a syringe.

For purposes of the present invention, the term “trocar needle” refers to a medical device that is made up of an obturator with a sharpened non-bladed tip, a hollow tube surrounding the obturator and a seal.

### Description

While the present invention is disclosed with references to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof. It is understood that other embodiments may be utilized and structural changes may be made without departing from the scope of the invention.

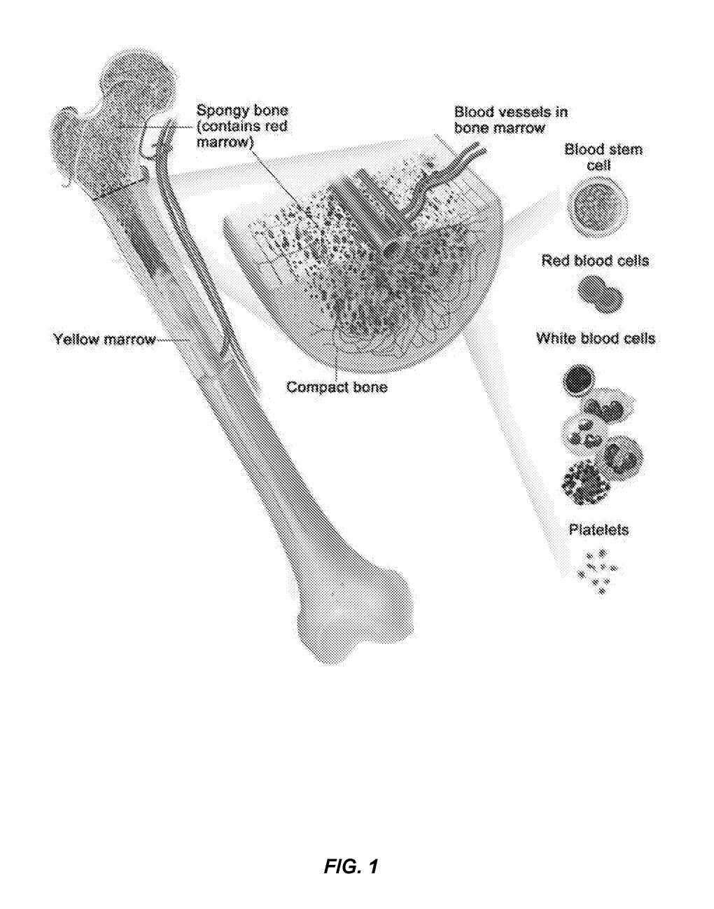

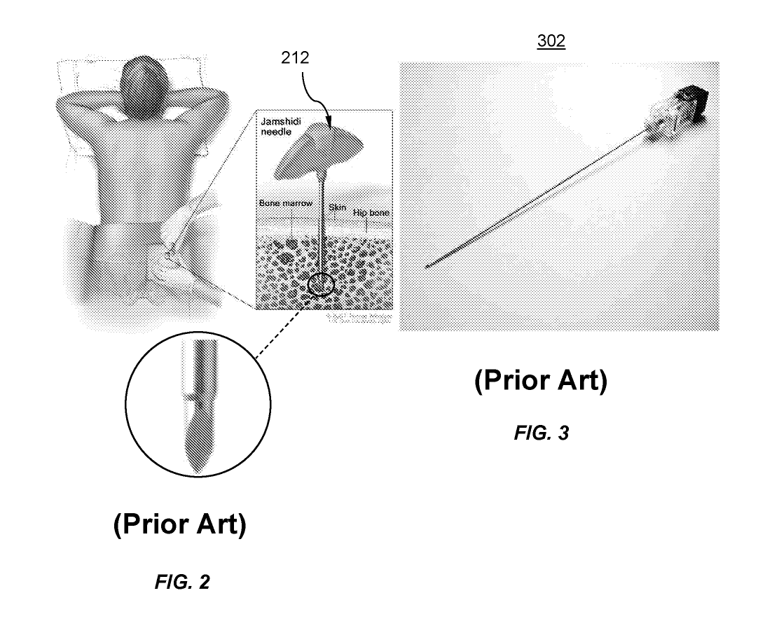

FIG. 1 shows the structure of a human femur (thigh bone) including a compact cortex on top of a spongy bone layer containing the marrow and the blood cells that are produced by and replenished from the marrow. FIG. 2 shows a bone-access process that involves inserting a conventional biopsy needle 212 through the soft tissue and the compact cortex layer into the region holding the bone marrow. The standard procedures for performing bone marrow biopsy and aspiration involves palpitation of the patient's posterior iliac bone to locate a target site for the procedure. Once the target location for the biopsy/aspiration has been roughly determined, an anesthetic such as lidocaine is administered in the skin, typically, for example, with a 25 gauge (G) needle, such as needle 302 shown in FIG. 3. A spinal needle is then advanced through the anesthetized skin without guidance until it hits bone. A common local anesthetic such as lidocaine is administered at the bony surface where the bone must be penetrated for retrieval of bone marrow specimen. Bone marrow sample extraction is then performed by making an incision at the surface of the skin and inserting a bore trocar needle, consisting of an inner or central needle with a sharp distal tip (called a stylet), for boring through the cortex, and a surrounding sheath (called a cannula), through the patient's soft tissue down to the approximate site of the anesthetized bone surface. Once the needle-lined sheath reaches the bone, the needle is manually cored into the bone using a rotating motion, similar to driving a screw, until it pierces through the hard outer covering of the bone (cortex). Once the needle is inside the bone, the stylet is pulled back through the sheath (cannula), and fluid marrow aspiration is performed by attaching a syringe to the opening at the proximal end of the hollow needle (cannula) in order to retrieve a sample of liquid bone marrow.

Typically two aspiration steps are performed to obtain two fluid marrow samples with and without Ethylenediaminetetraacetic Acid (ETDA). This means that after the first aspiration step with ETDA the syringe must be removed from the opening at the proximal end of the needle and another syringe placed on the needle to perform dry syringe aspiration. Bone marrow aspiration induces pressure changes in the marrow space which may cause severe pain for some patients, even with intravenous (IV) pain medication and conscious sedation (which are generally not utilized in an office setting). After a fluid marrow specimen has been retrieved, the hollow needle is then “cored” through the trabecular bone (spongy bone holding the marrow) in an attempt to retain a piece of solid marrow in the cannula needle. The needle is then rocked back and forth to “break off” the end of the cored piece of trabecular bone and separate it from the surrounding marrow such that it remains in the needle as the needle is withdrawn. The hollow needle is then removed, bringing with it the collected core sample.

A major source of pain and discomfort may stem from potential misalignment of the bone drilling/coring tip of the stylet with the anesthetized bone surface that received the numbing agent. Since bone aesthesis is performed during the first pass to the bone (e.g., lidocaine being delivered to the bone surface via a spinal needle) and bone penetration for marrow sample extraction is performed during the second pass, the hematologist, pathologist or other operator performing the procedure must approximate as accurately as possible the site of the anesthetized bone surface that received the lidocaine during the first pass. Depending on the accuracy and dexterity of the hematologist, lidocaine may or may not be acting at the site of drilling/coring (current procedure usually dictates coring device rather than drill). If the drilling/coring of the cortex is not at the location where the spinal needle delivered the anesthetic, it can cause excruciating pain for the patient.

Sometimes patients find it difficult, or cannot tolerate the above procedure, or have had complications of the blind placement of the needle such as puncture of the dural sac that covers the spinal cord. Therefore, upon request of the patient, the procedure is sometimes performed by a radiologist with conscious sedation and computed tomography (CT) image guidance. This necessitates the patient not eating for a prescribed time period (such as, for e.g., 8 hours), an expensive nursing staff to monitor the procedure and deliver drugs, and adds the risk of IV anesthesia and the cost of a recovery room staff. It also adds the expense and necessitates access to a CT scanner and a Technologist and nursing staff. Additionally, CT guidance has limited access in many parts of the world and may subject the patient to unnecessary exposure to radiation.

Furthermore, the needle position is not visualized in real time while it is being advanced. This means that, in conventional procedures, in addition to the initial scan of the patient with a grid table covering the scanned area to mark the site of skin entry, multiple additional scans may be required to check the position of the spinal needle in relation to the target bone site while making necessary alteration to the angle and placement of spinal needle as it is advanced towards the target site. This process is repeated until the needle ends in the right place by trial and error. Upon administration of the anesthetic at the surface of the bone and removal of the spinal needle, a biopsy/aspiration needle is placed into the tissue and advanced and checked in the same way as the spinal needle until the surface of the bone is reached by trial and error. Using image guidance may facilitate drilling/coring of the cortex at the location on the surface of the bone where the anesthetic is administered, but this is not always the case.

Furthermore, the cortex penetration is typically performed manually wherein a handle placed on the needle is grasped, for instance, in the palm and twisted back and forth to forcibly dig/burry the needle through the cortical bone. Generally, the motion of the wrist does not naturally twist back and forth in a straight line like a drill, but rather, moves in an arc, thereby, causing more discomfort for the patient, especially if it is not at the location of anesthetic placement. Alternatively, a power drill may be placed on the needle in order to drill the needle through the cortex to expedite the cortex penetration process. However, regardless of whether a grasp handle (for manual coring) or a power drill (for drilling through the cortex) is placed at the proximal end of the needle, the contraption must be taken off the needle every time the stylet is removed or a syringe is placed on the needle to obtain a marrow aspirate and subsequently placed back onto the needle to advance the needle through the marrow in order to obtain a core biopsy sample.

It is, therefore, highly desirable to reduce the pain associated with bone marrow biopsy while expediting the procedure to reduce the discomfort and suffering experienced by the patient, both in duration and intensity of pain, while requiring less time and effort by the medical professional to perform and complete the procedure. Present embodiments of the disclosed invention provides a method and apparatus for an improved biopsy device with integrated functionality capable of combining multiple steps into one to, therefore, perform the procedure with increased precision, in less time and with less pain.

In one embodiment, the present invention provides a method and apparatus for an improved biopsy device with integrated functionality capable of: (1) combining multiple steps, (2) providing low cost image guidance and (3) allowing for anesthesia of the entire bone, which together will allow the procedure to be performed with increased precision, in less time and with less pain.

One aspect of the present invention addresses the issue of precisely aligning a prescribed drilling site and an anesthetized surface region by performing both tasks (e.g., bone drilling and bone anesthesia) during a single operational procedure. Thus, in one disclosed example, alignment of a prescribed drilling site and anesthetized bone surface region is performed during a single procedural needle trip to the bone. One embodiment of the present invention discloses a biopsy needle with an integrated anesthetic delivery mechanism wherein spinal needle functionality is effectively integrated into the bone drill needle in order to anesthetize and extract marrow sample in a single pass to the bone, resulting in inherent alignment of anesthetized site and bone drill site.

In one embodiment, the present invention provides a device and a method for integrating anesthetic delivery to the soft tissues, periosteum (covering of the bone) and intraosseous (within the bone marrow cavity), as well as bone marrow extraction into a single simultaneously performed operation using a multi-functional biopsy device with an automated ultrasound guidance and navigation system.

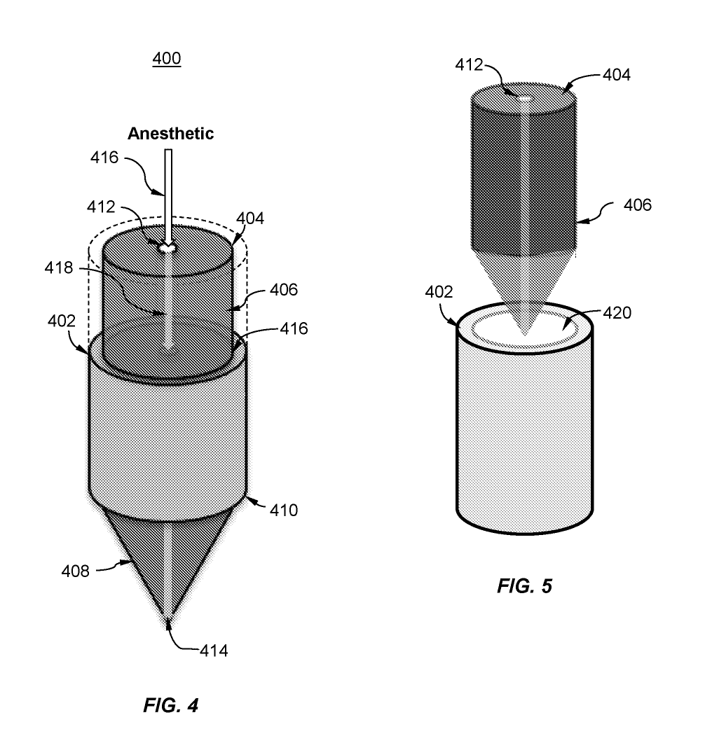

FIGS. 4 and 5 illustrate an embodiment of a needle device according to one embodiment of the present invention. Needle device 400 in FIG. 4 comprises a hollow tubular sleeve 402 encompassing a (removable) central stylet 404. The (removable) central stylet may be referred to as a central stylet, removable stylet, stylet or inner needle. The hollow tubular sleeve may be referred to as the cannula or the outer needle. Central stylet 404 comprises a coaxial shaft 406, a sharp distal tip 408 that extends past end-surface 410 of hollow tubular sleeve 402 and is used for penetrating through the cortex, and a hollow central conduit 412 that extends coaxially through central stylet 404 and opens to a portal 414 through which an anesthetic, such as lidocaine, indicated by arrows 416 and 418 may be delivered directly to the bone surface at the target biopsy site. Placing a hollow central conduit 412 into the middle of removable central stylet 404, essentially integrates an anesthetic delivery channel into the stylet, thus, consolidating the previously required two needle trips to the bone into a single pass while ensuring that anesthetic is placed at the breach site of the application location, such as the cortex. The hollow central conduit may be referred to as an anesthetic delivery channel, central transport channel, hollow transport channel or transport channel in the context of one or more embodiments of the present invention. It should be noted that the central stylet may comprise of any number of hollow central conduits extending coaxially along any longitudinal axis within its volume. In addition, the location of the central stylet is not restricted to the central longitudinal axis of the central stylet. Furthermore, the hollow conduit may be used as a general transport channel, for example, for delivering one or more therapeutic agents onto a target biopsy site or for retrieving one more biopsy specimens from a target biopsy site. In one disclosed embodiment, the hollow central conduit disposed within a central stylet of an exemplary biopsy needle may be used as an anesthetic delivery channel—more specifically, for delivery of lidocaine to the bone surface of a target biopsy site.

The exemplary biopsy needle structure, illustrated in FIG. 4, may be used for a single-pass bone-marrow specimen extraction. An anesthetic agent, such as lidocaine, may be delivered to the surrounding soft tissue for anesthetizing the path of needle device 400 as it is being advanced toward the target bone surface location. Once at the target bone surface, the bone drill site may be anesthetized by the delivery of the numbing agent, such as lidocaine, through hollow central conduit 412. The hollow central conduit may be referred to as an anesthetic delivery channel in the context of the present invention and its location may not be restricted to central longitudinal axis of the stylet or the inner needle. The delivery of anesthetic at the location of portal 414 disposed within a sharp distal tip 408 of central stylet 404, overlaps the site where the bone surface is breached as both procedures are performed at the tip of the same needle element. As sharp distal tip 408 of central stylet 404 penetrates the hard cortex, anesthesia, for example lidocaine, may be delivered directly into the marrow cavity through an anesthetic delivery channel, i.e., hollow central conduit 412. With the hard surface of the bone breached, the lidocaine may be infused into the intramedullary space for numbing the pain that may result from the pressure change in the marrow cavity during the aspiration process. The retraction of central stylet 404 from the biopsy needle leaves behind a hollow channel cavity 420, disposed within the outer needle, to which a syringe (not shown in FIGS. 4 and 5) may be attached in order to extract a sample of fluid marrow aspirate through the hollow channel into the syringe. In addition, hollow channel cavity 420 provides a containment space for receiving a solid marrow core sample when needle device 400 is driven into the marrow cavity.

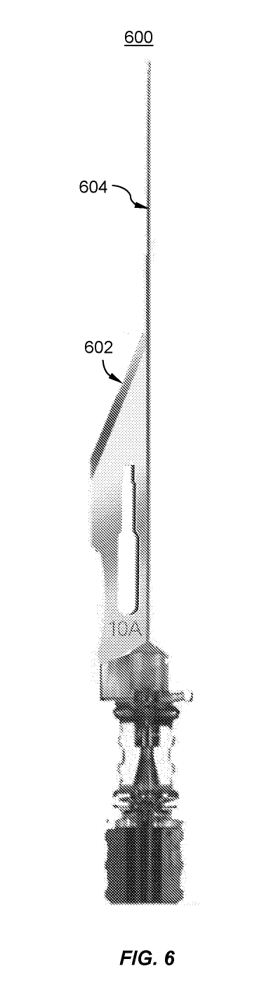

FIG. 6 illustrates a biopsy needle 600 according to one embodiment of the present invention comprising a skin cutting blade 602 in combination with an anesthetic delivery needle 604 for optimizing percutaneous procedures that require a dermatotomy. In one embodiment of the present invention, a number 10 blade is used as dermatotomy (skin cutting) blade 602. Biopsy needle 600 enables anesthesia delivery to the skin and subcutaneous tissues, as well as the dermatotomy to be performed during a single procedural pass without a need to change instruments. This may not only save both time and effort for the procedure, but may also significantly reduce any discomfort to a patient.

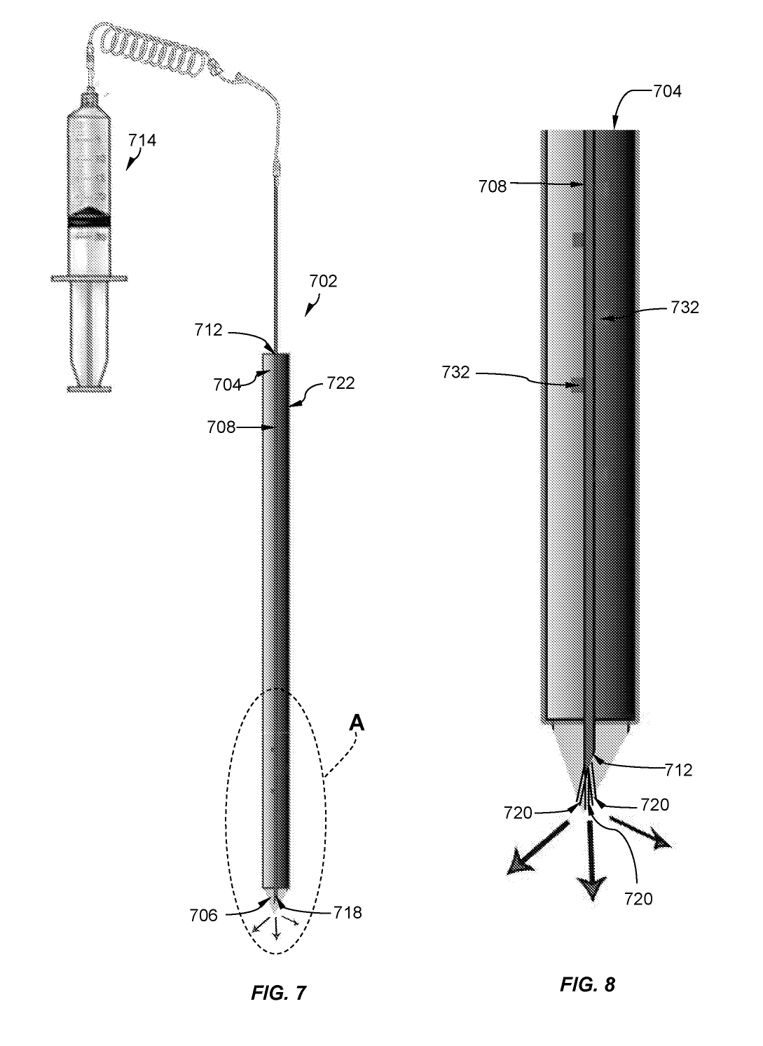

In one aspect of the present invention, a transport channel is axially integrated into a procedural needle such as a biopsy needle. FIGS. 7, 8, 9, 10, 11 and 12 illustrate an exemplary embodiment of a biopsy needle comprising a central transport channel for use in soft tissue biopsy or bone marrow biopsy. An exemplary biopsy needle 702 is illustrated in FIG. 7. Biopsy needle 702 includes an inner needle 704 with a sharp distal tip 706 for easy advancement through tissue material and a central transport channel 708. Central transport channel 708 includes a proximal end with an opening 712 to which a syringe 714 may be connected and a distal end with an opening 718 that is connected to end-openings 720 disposed at sharp distal tip 706 of inner needle 704. Central transport channel 708 also includes side openings 732.

Although multiple end-openings are shown in FIGS. 7 and 8, in some embodiments of the present invention, there may only be a single end-opening in the sharp distal tip of the needle.

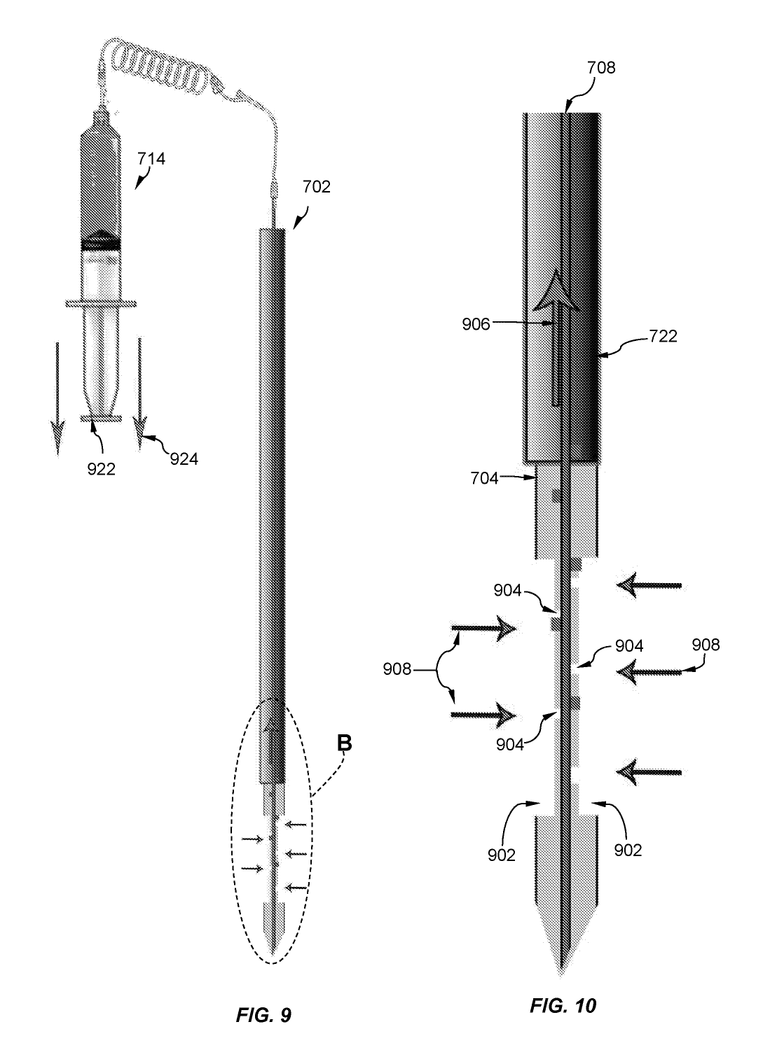

Inner needle 704 may include a sampling trough region 902, as illustrated in FIGS. 9 and 10. Inner needle 704 is ensconced by a coaxial hollow outer sleeve 722. Inner needle 704 and outer sleeve 722 may be moved in an axial direction relative to one another, for example, by retracting outer sleeve 722 by pulling outer sleeve 722 back from inner needle 704 or advancing forth inner needle 704 out of outer sleeve 722. In FIG. 7 outer sleeve 722 is shown in a down position, thus, in FIG. 7 sampling trough region 902 is covered by outer sleeve 722.

In one embodiment of the present invention sampling trough region 902 of inner needle 704 may comprise a plurality of side openings 904 which connect to central transport channel 708, thus enabling, for example, administration of an anesthetic agent, such as lidocaine, both from the end and the sides of the needle. In FIGS. 7 and 8 outer sleeve 722 is shown in an extended position, thus, covering side openings 904 in sampling trough region 902, which diverts anesthetic, such as lidocaine, from side openings 904 to end-openings 720 at the tip of inner needle 704. This enables the anesthetic to be delivered at the tip of the needle. Referencing FIGS. 9 and 10, during the next step of the operation of the biopsy needle 702, after anesthesia has been administered through sharp distal tip 706, side openings maybe unveiled as outer sleeve 722 is retracted, or inner needle 704 is advanced out of outer sleeve 722. At this point, side openings 904 in sampling trough region 902 may be used for providing a suction force, in the direction noted by arrow 908, for pulling a maximum amount of tissue into the sampling trough region to ensure an adequate specimen. In FIG. 9, arrows 908 denote the direction of suction force on the tissue sample surrounding sampling trough region 902. The suction may be provided through the same syringe, i.e., syringe 714, which applied the anesthetic by pulling piston 922 in the direction of arrows 924.

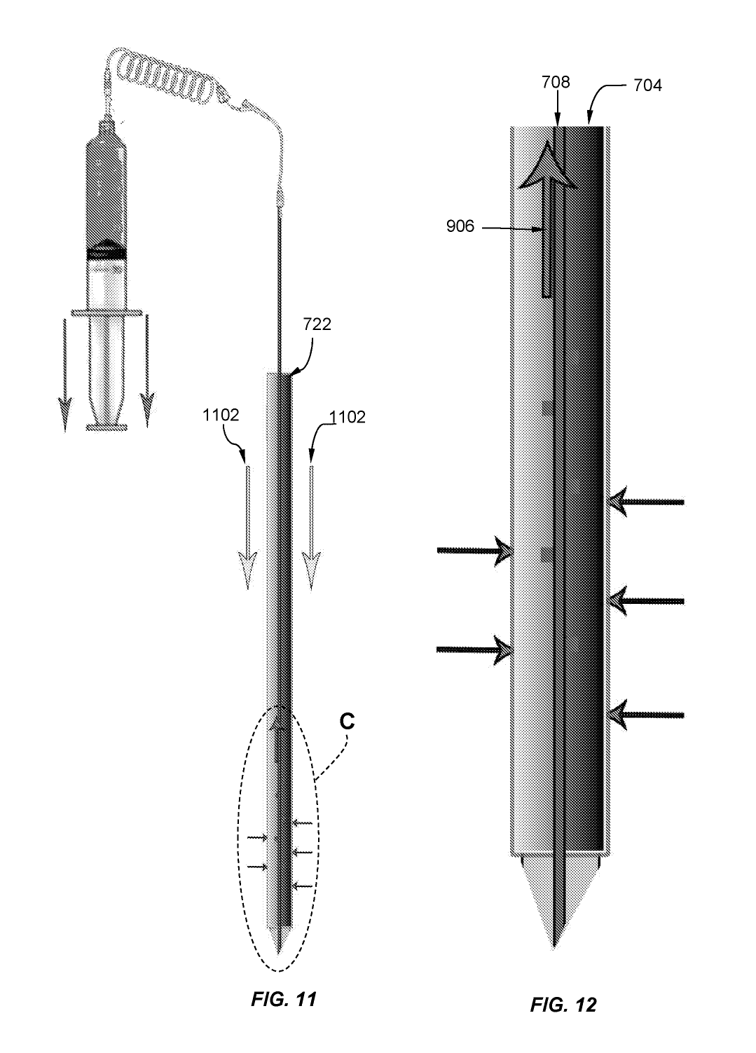

FIGS. 11 and 12 illustrate additional features of procedural biopsy needle 702, where the outer sleeve is fired quickly over the top of inner needle 704 and sampling trough region 902 to cut a piece of tissue specimen and retain it into sampling trough region 902. Arrow 1102 in FIG. 11 illustrates the forward motion of outer sleeve 722 while suction force 906 is applied through central transport channel 708. In a different embodiment, specimen tissue retrieval may be performed with or without the addition of suction as described above.

In some embodiments of the present invention, the motion of the outer needle comprising retraction from inner needle 704 followed by forward advancement over inner needle 704 that facilities acquisition of tissue sample is spring-loaded. In other exemplary embodiments, the mechanical lever used to provide suction through side openings 904 disposed within sampling trough region 902 are linked to the mechanical levers to spring load and fire outer sleeve 722 so the process of tissue sample collection may be performed in a single motion.

The exemplary embodiment, illustrating utility of the central transport channel as: (1) an anesthetic delivery channel for anesthetizing the surface of the skin, (2) a path of the needle through the skin toward the target biopsy site and the target biopsy site, and (3) a suction channel to facilitate collection and retrieval of a soft tissue specimen, such as soft, necrotic or gelatinous/mucinous tumors.

Although in some described embodiments the term “central” is used in conjunction with the term “transport channel,” i.e., “central transport channel,” it is readily appreciated that the placement of the transport channel is not restricted to the central axis of the inner needle or the central stylet. Furthermore, it is not intended to limit the embodiments of procedural devices, i.e., soft tissue and bone marrow biopsy needles, to restrict the number or dimensions of disclosed features such as the transport channel, sampling trough region, etc.

The exemplary biopsy needles and stylet structures illustrated in FIGS. 4, 5, 6, 7, 8, 9, 10, 11 and 12 may be used as a one pass bone-access device to provide local anesthesia, extract a solid marrow core sample and fluid marrow aspirate, with a single entry into the bone. The needle assembly illustrated in these examples may have an anesthetic, such as lidocaine, connected to it so when a stylet is retracted, lidocaine is drawn into the marrow space rather than air, as air may possibly cause an embolism if injected into the marrow space. The integration of a transport channel for delivery of a therapeutic agent also enables intraosseous administration of the anesthetic through the biopsy needle so that the aspiration is not painful.

In one example, a 1 cc saline flush will clear the needle of the anesthetic. The exemplary embodiments illustrated in FIGS. 4, 5, 6, 7, 8, 9, 10, 11 and 12, described herein, are not meant to restrict device parameters such as the number or dimensions of the central stylets or the surrounding outer needle structure and the number of anesthetic delivery channels in the embodiment of biopsy needle disclosed in the present invention. One or more biopsy/aspiration channels comprising of one or more central stylets and one or more outer needle structures may have different cross-sectional diameters depending on the application or procedure for which they are used. Furthermore, it should be noted that biopsy needle embodiments may comprise one or more integrated transport channels.

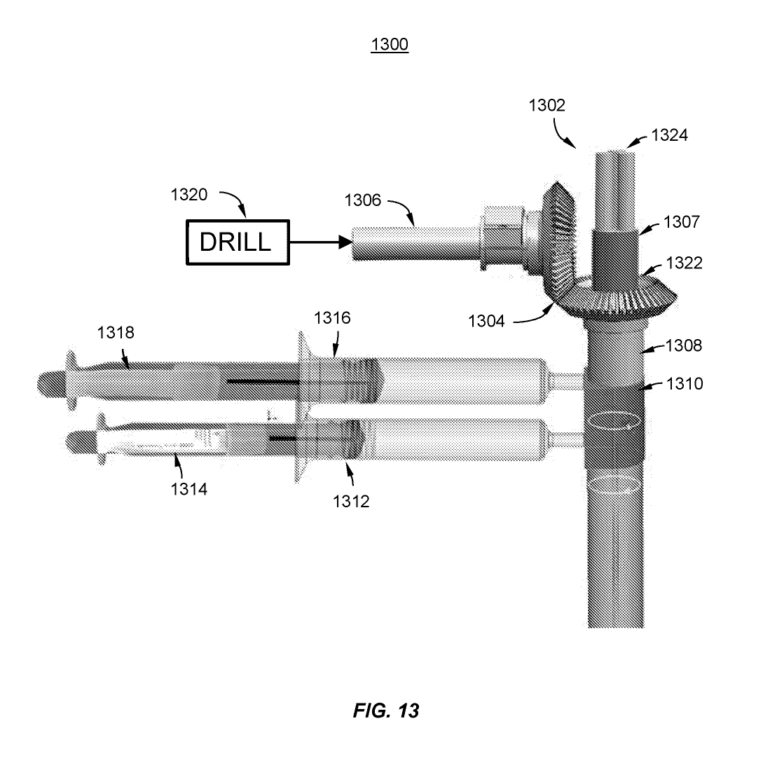

In addition to the one-pass lidocaine delivery and bone marrow sample retrieval functionality, further measures of the disclosed invention are implemented to speed up the biopsy process and to eliminate additional unnecessary steps. FIG. 13 illustrates one embodiment of an integrated painless biopsy device in accordance with the present invention. Needle assembly 1300 comprises a main bone-access needle 1302, a right angle gear 1304 and a drill connection shaft 1306, serving as a mechanical drill interface, a central stylet 1307 disposed within an outer sleeve 1308. Outer sleeve 1308 comprises a slip joint 1310 with a stationary outer portion 1311 for connection to one or more syringes. Needle assembly 1300 further comprises an aspiration syringe 1312 containing, for example, an anticoagulant (such as EDTA) with an integrated sterile vacuum tube 1314 and an empty aspiration syringe 1316 with an integrated sterile vacuum tube 1318 containing Heparin.

Right angle gear 1304 serves as a mechanical drill interface to transfer the rotation of drill 1320 to main bone-access needle 1302 without obstructing the opening at a proximal end 1322 of main bone-access needle 1302. By allowing proximal opening 1322 of main bone-access needle 1302 to remain accessible, the right angle configuration obviates the need to remove and re-attach the drill every time a stylet is removed. Current drills do not allow access to the needle, therefore the drill must assemble on and off multiple times, thereby, lengthening the biopsy procedure and at times, moving the drill bit. This may also induce unnecessary discomfort and pain to the patient during the biopsy procedure.

Central stylet 1307 may comprise one or more perforated connections 1324. It is readily implied that perforated connections 1324, shown as pillars in FIG. 13, may be of any configurable shape and dimensions within central stylet 1307. The perforated connections demonstrate the communication of the one or more syringes attached to the body of main bone-access needle 1302, via slip joint 1310, with central stylet 1307, which may be disposed within slip joint 1310. This, in conjunction with slip joint 1310, which provides side-access to an inner lumen of central stylet 1307, and an inner lumen of outer sleeve 1308, allows the one or more syringes, for example aspiration syringes 1312 and 1316, to connect to an inner lumen of central stylet 1307 and an inner lumen of outer sleeve 1308, simultaneously. Side-access enabled configuration, in addition to leaving the opening at the proximal end, i.e., proximal opening 1322 of main bone-access needle 1302 unobstructed, enables parallel access to main bone-access needle 1302. Therefore, additional prescribed accessories may be attached to main bone-access needle 1302 either at once or as desired.

As shown in FIG. 13, slip joint 1310 allows multiple aspiration steps to be consolidated into one by enabling, for example, simultaneous draws of heparin and ETDA into aspiration syringes 1312 and 1316. A one-way valve may be placed at the end of the EDTA syringe, so that upon aspirating the dry syringe, it does not suck the EDTA out of the EDTA syringe. Slip joint 1310 also enables the syringes to remain stationary while connected to stationary outer portion 1311 of slip joint 1310, thereby, allowing the needles to spin with the drill. Sterile vacuum tubes enable press-fitting transfer samples into the respective containers, which may decrease the chance for a clotted sample, decreases the likelihood of the operator inadvertently breaking sterile procedures while passing off the syringe, and decrease the length of the procedure. One disclosed embodiment comprises a needle assembly comprising a pre-attached drill and includes a set of syringes already attached to the needle assembly. In selected embodiments, the pre-attached drill may include a permanent configuration. The pre-attached syringes may include one of several types, for example, including a lidocaine syringe, a saline flush syringe, an EDTA aspiration syringe and a dry syringe for heparin aspiration.

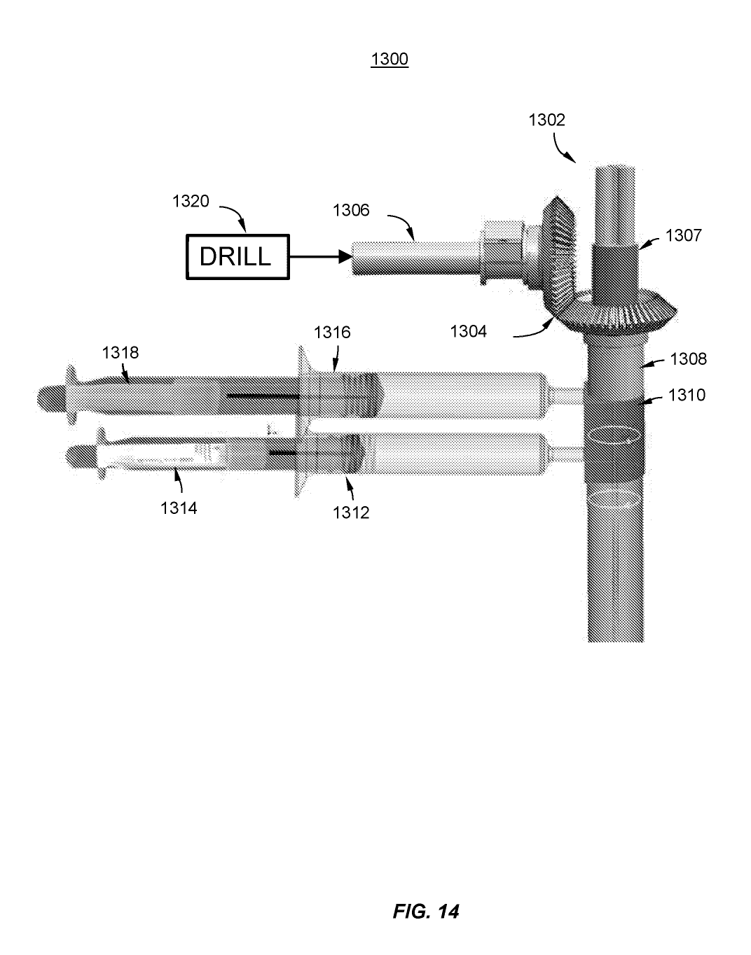

FIG. 14 illustrates an exemplary biopsy needle 1400 performing simultaneous aspirations of two bone marrow specimens 1402 and 1404, drawn through hollow channel 1406 disposed within outer needle 1408 when central stylet 1410 is retracted. In the exemplary biopsy needle, i.e., biopsy needle 1400, two aspiration syringes 1412 and 1414 connect to hollow channel 1406 which is used for aspiration and/or marrow core sample retrieval.

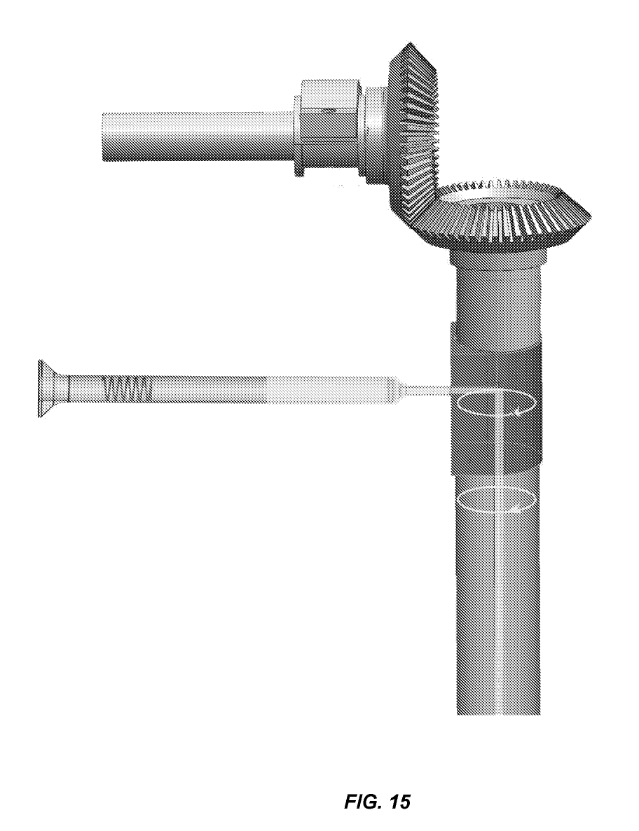

FIG. 15 illustrates an anesthetic syringe (e.g., a lidocaine syringe) 1502 connecting to inner lumen 1504 disposed, for example, within a central stylet (not shown in FIG. 15). The anesthetic syringe and the aspiration syringe may be loaded separately or simultaneously onto the biopsy needle, example illustrated in FIG. 14 and FIG. 15.

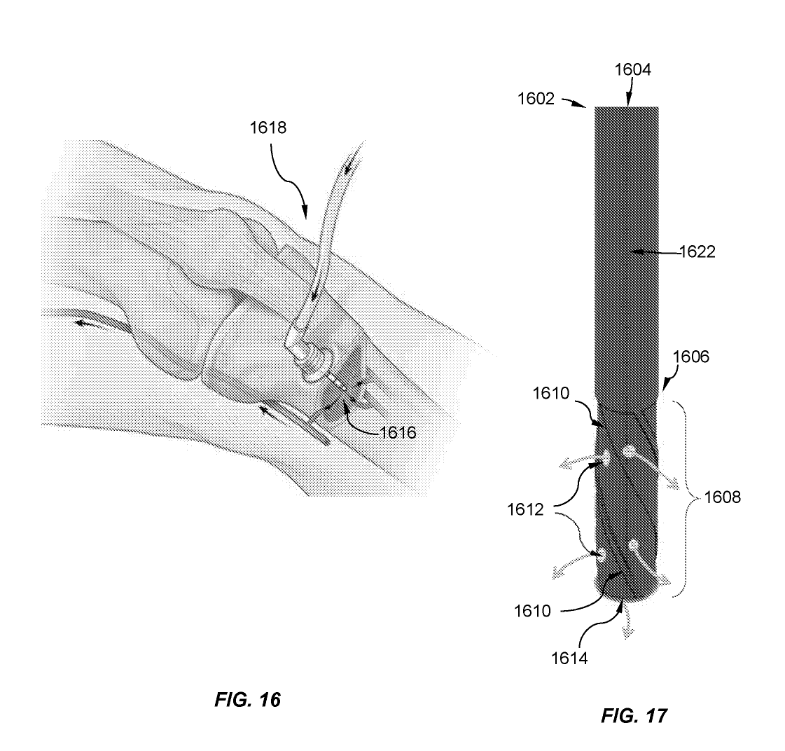

A standard bone-access needle may include the same cross-sectional diameter as the bore that is drilled by its tip. This may cause binding in the cortex, especially when going through young or sclerotic bone. Facilitating an easier passage through the bone can further lessen any discomfort and the time associated with bone marrow biopsy and aspiration. Therefore, in one embodiment of the present invention, a cutting edge optimized for drilling bone with reduced binding when breaching the cortex is added to the distal end of the needle assembly. FIGS. 16 and 17 show exemplary embodiment of a biopsy needle drilling section 1602 comprising an integrated anesthetic delivery channel 1604 and a distal end 1606 that is optimized for drilling bone. The optimization comprises modifying distal end 1606 of biopsy needle drilling section 1602 with a prescribed pattern. In one disclosed example, a groove pattern is provided to cut a diameter through the bone slightly larger than the diameter of needle distal end 1606 and has been modified into drill bit portion 1608. In one embodiment of the present invention, a helical groove pattern 1610 is selected for this purpose.

Disclosed embodiments of drill bit portion 1608 may comprise end and side holes that allow an anesthetic agent, for example lidocaine, to disperse there-through. Anesthetic delivery channel 1604 extends through drill bit portion 1608, allowing the anesthetic agent to be dispersed outward in both lateral and downward directions, through side holes 1612 and end holes 1614, into intraosseous space 1616 as shown by illustration 1618 in FIG. 16. Therefore, the disclosed drill bit structure allows for optimized bone drilling while enabling bone surface aesthesis and intraosseous anesthetic infusion, in a single procedural pass to the bone biopsy site.

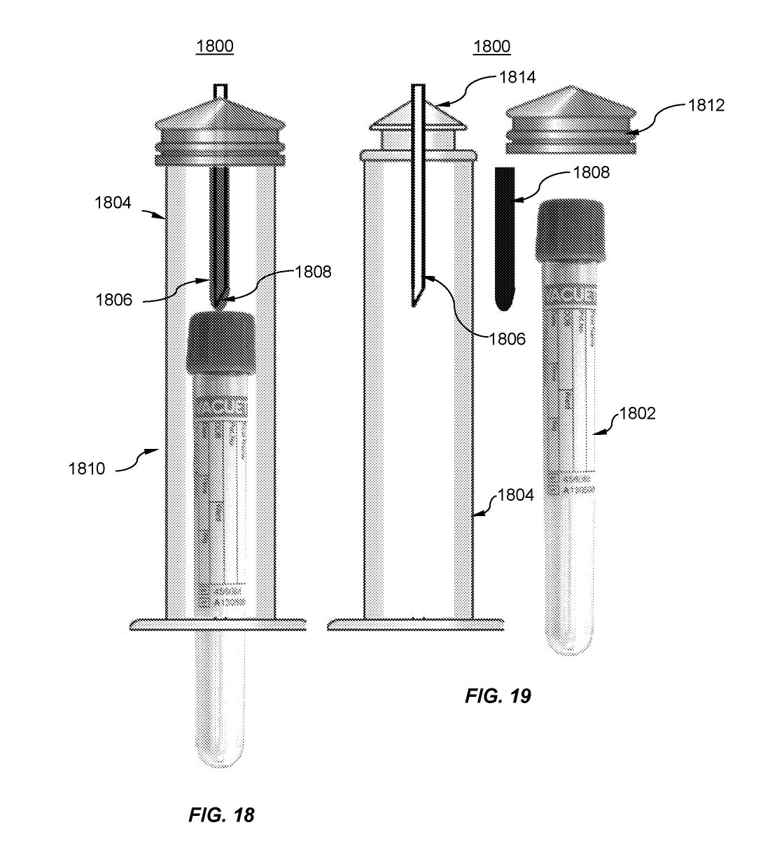

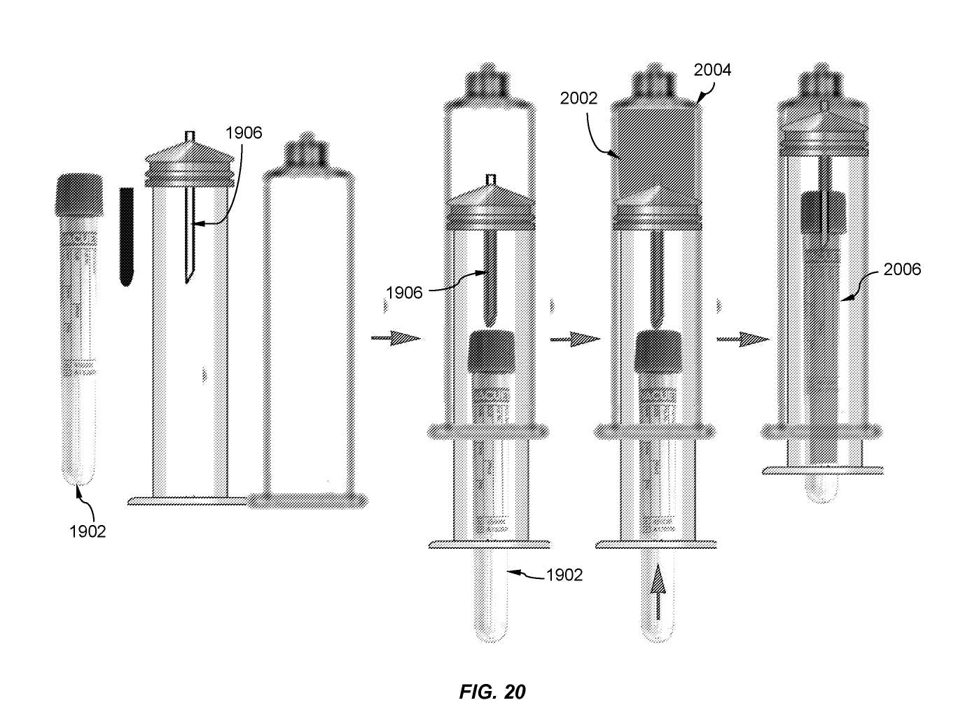

FIGS. 18 and 19 illustrate a novel aspiration syringe plunger/syringe configuration 1800 with vacuum tube 1802 fitted within plunger 1804. Plunger 1804 includes a needle 1806 that traverses a rubber part 1808 of plunger 1804 into syringe 1810. Plunger 1804 includes a cap 1812 that fits on a plunger body 1814. Needle 1806 may be covered with rubber on the end near the vacuum tube. The disclosed syringe/plunger system allows for the transfer of specimen quickly and without a second non-sterile person to assist. This procedure is illustrated in FIG. 20. When vacuum tube 1802 is pressed over the rubber, needle 1806 is exposed within vacuum tube 1802 and vacuum tube 1802 sucks out bone marrow 2002 from syringe 2004, transferring bone marrow 2006 into vacuum tube 1802.

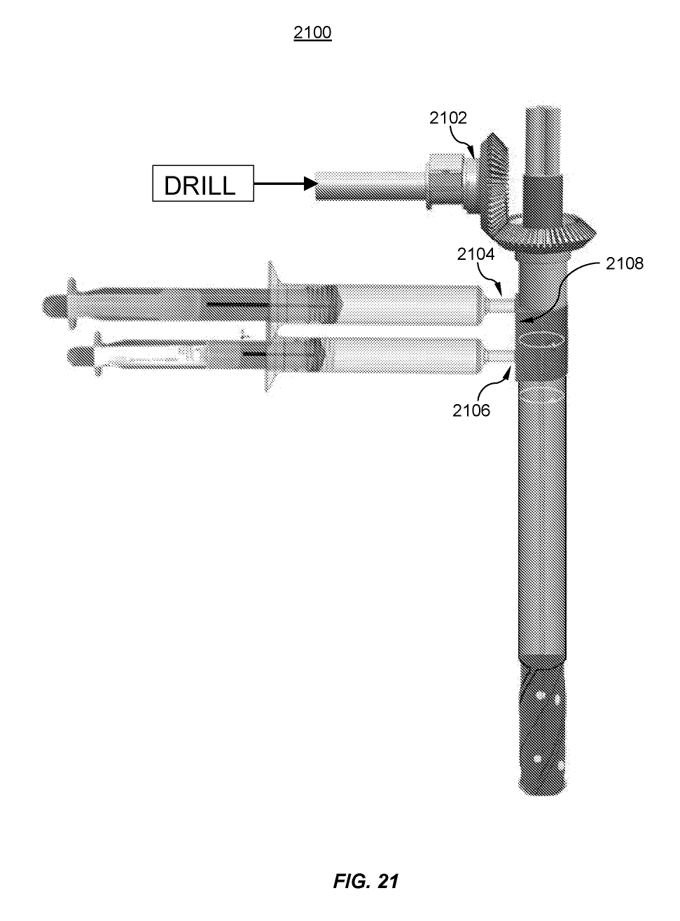

FIG. 21 illustrates one exemplary embodiment of an integrated one-pass biopsy needle assembly 2100 with a right angle drill interface 2102, side-access ports 2104 and 2106 with stationary cover 2108 for multiple attachments of anesthetic and/or aspiration syringes and a distal drill bit 2110 optimized for bone drilling, and bone surface and intra-osseous lidocaine dispersal, according to an embodiment of the present invention.

In another embodiment, the present invention describes a portable user-friendly and cost-effective ultrasound needle guidance system. The ultrasound needle guidance system may render image guided bone-access in accordance with an accessible procedure with minimal training. This may be accomplished in part by minimizing user intervention through full or partial automation of procedural steps that would normally require a high degree of skill and experience to perform. In one embodiment of the present invention, the ultrasound needle guidance system utilizes automatic processing of the captured ultrasound to optimize depth and contrast for the procedure. Embodiments may include utilizing software for automatic processing of the captured ultrasound in order to optimize contrast and tailor the depth, field of view and frequency of signal, e.g., automatically, for the user. The ultrasound needle guidance system software may also further tailor the ultrasound image by filtering the image for best visualization of the bone and the needle. This may involve enhancing the signal from the strong reflector of sound, such as bone and needle, while attenuating the signal from all other intervening tissue.

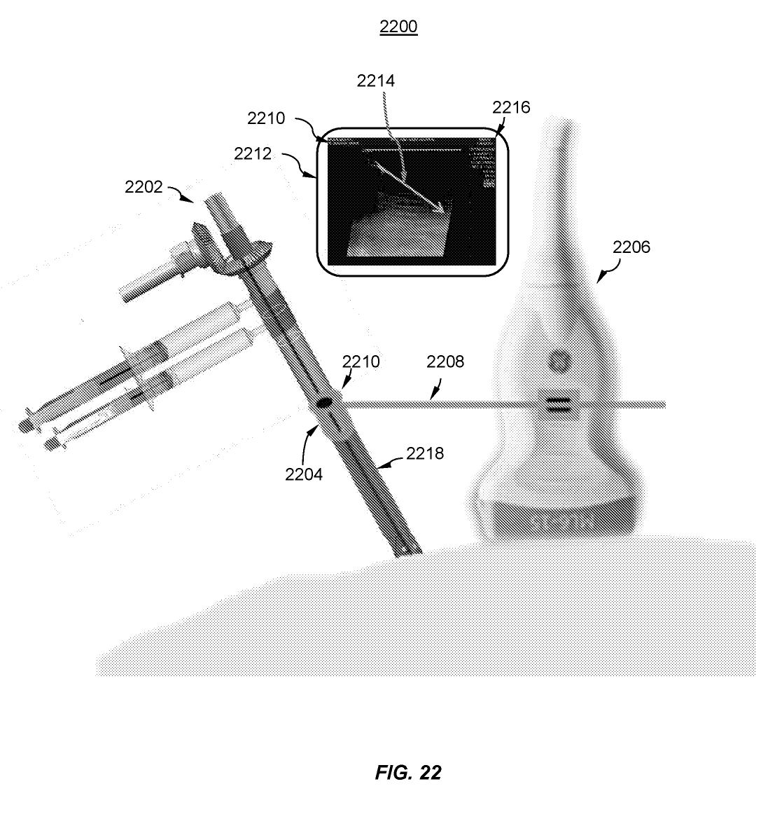

FIG. 22 illustrates one exemplary embodiment of an ultrasound-guided integrated painless biopsy system 2200 comprising an integrated biopsy device 2202 inserted in a lockable, articulating needle guide 2204 that attaches to an ultrasound probe 2206 across a translation arm 2208 and a rotation dial 2210. Biopsy system 2200 is designed to incorporate painless one-pass biopsy device 2202 with an ultrasound guidance system that may be used to predict a needle trajectory and to control the movement of a needle. In a select embodiment, biopsy device 2202 may employ similar features of the biopsy device shown in FIG. 13 and FIG. 21, described above, and as described below. Additionally, biopsy system 2200 comprises a display screen 2212 that provides real-time visualization of predicted needle trajectory 2214 overlaid on ultrasound image 2216. As needle guide 2204 is moved and angled, an indicator, such as predicted needle trajectory 2214, is drawn on display screen 2212 that represents the trajectory the needle will take. Guide 2204 may be adjusted until the best insertion angle and puncture line corresponding to the most optimal needle path to the target bone surface is chosen. Needle guide 2204 has a laser 2218 that shines on the skin, indicating the correct skin entry point. This obviates the need for the standard indelible ink for marking an entry point. Needle guide 2204 is then locked into position and will be rendered immovable in order to prevent needle deviation from the desired trajectory during insertion.

After the image of the biopsy site has been captured, the angle of the ultrasound beam generated by needle guide 2204 is adjusted automatically (or by user entered command) in order to image the trajectory of the needle as it is moved toward the target site. The captured image of the needle trajectory is overlaid onto the image of the biopsy site in real time, thus, providing an accurate and consistent real-time visual feedback of the needle trajectory for increased targeting accuracy. Enhancing needle visualization during the ultrasound-guided needle procedure requires that an optimal needle-ultrasound beam alignment be maintained. The best needle images are achieved when the needle is at right angles to the ultrasound beam. In order to accomplish this, the ultrasound beam may be dynamically steered, such as via software, in a direction perpendicular to the needle orientation for the entire length of the needle path to the target biopsy site.

In one embodiment of the present invention, as the needle guide translation arm 2208 is moved, the distance to the center of ultrasound probe 2206 is recorded as a length on the leg of a triangle. As needle guide 2204 is turned to draw trajectory 2214, the angle relative to needle guide translation arm 2208 is recorded electronically; this angle and this length are used to calculate trajectory 2214 across the screen.

In one embodiment of the present invention, a material that couples the ultrasound probe to fat, rather than water, may be utilized to achieve better penetration through fat and have an easier time on obese patients. Therefore, in one embodiment of the present invention a sterile fat-based lotion for a coupling material between the shell of the probe and the patient may be used instead of, for example, a water soluble gel. Additionally, as the tissue encountered in this part of the body for the bone marrow biopsy is strictly fat and bone, the ultrasound probe and speed of sound calculations, performed in software, will be optimized for the transit of sound waves in fat, which is approximately 1450 m/s, rather than the traditional ultrasound probes which are tuned for the speed of water, which is approximately 1497 m/s.

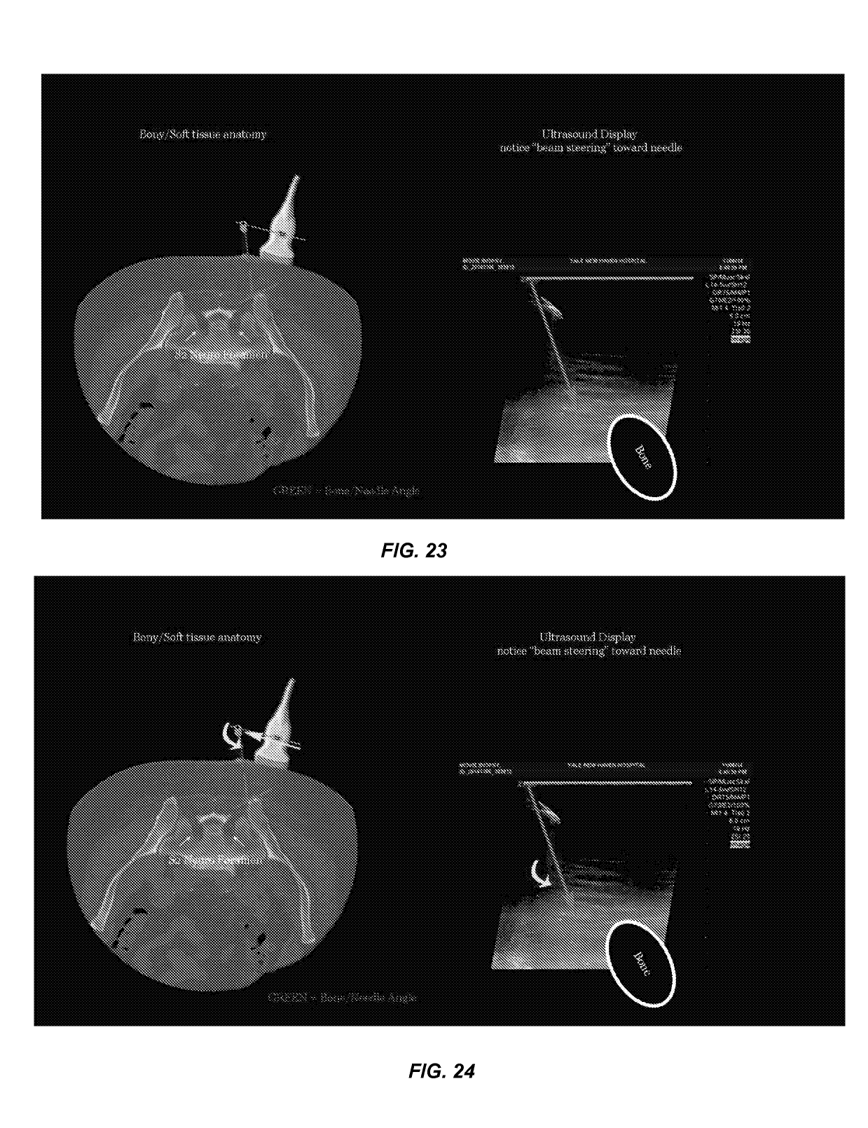

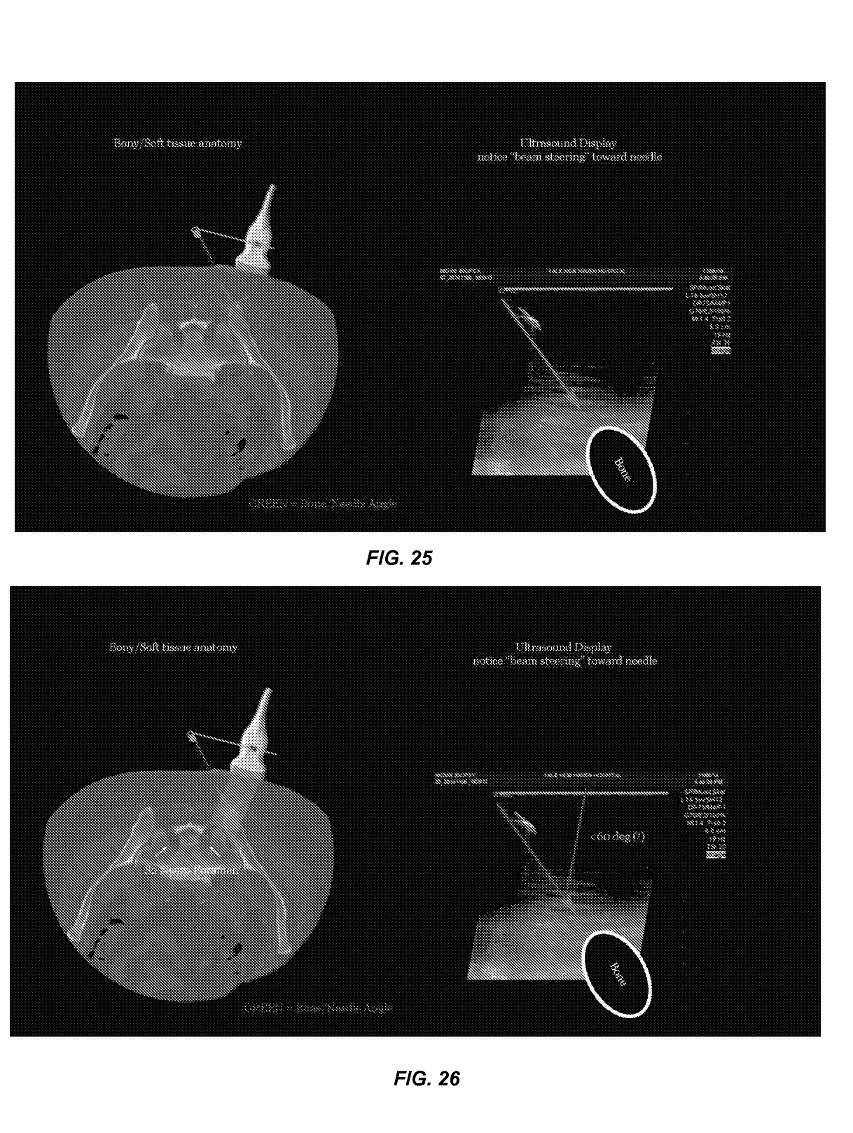

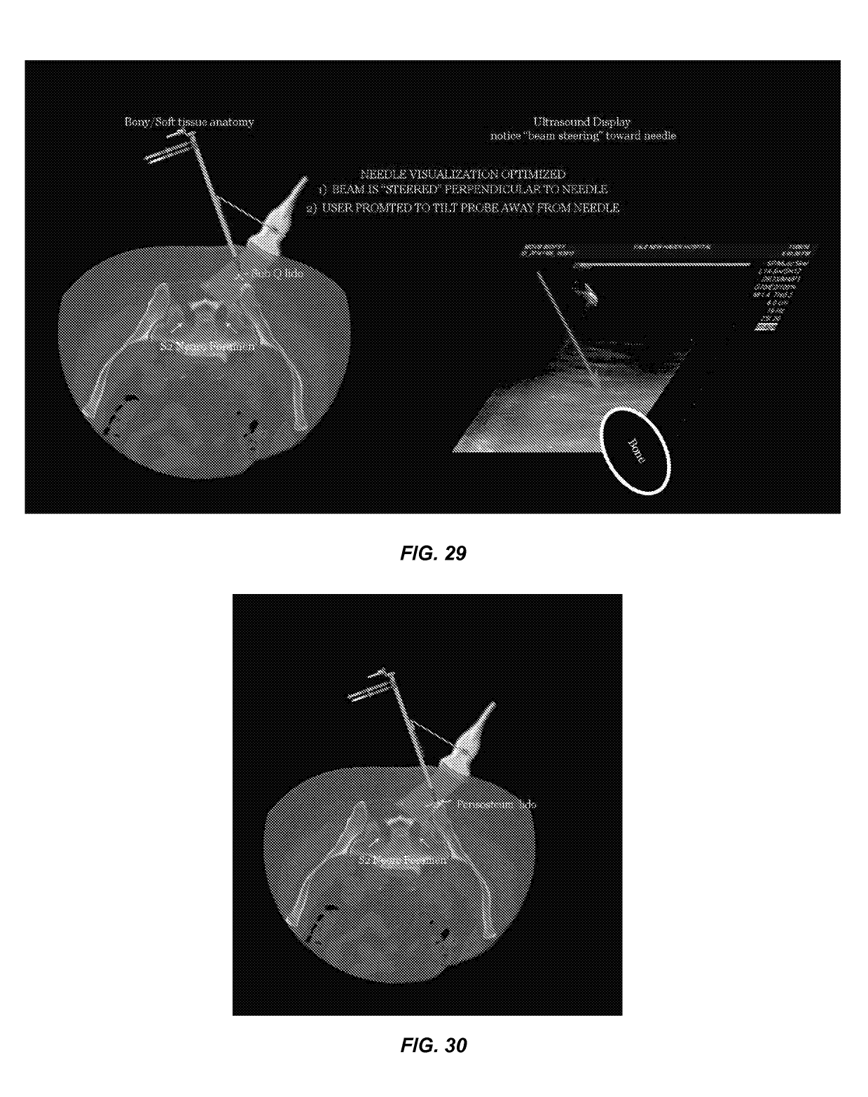

In FIGS. 23, 24, 25, 26, 27, 28, 29, 30, 31, 32, 33, 34 and 35, the ultrasound-guided painless one-pass biopsy procedure is described through several ultrasound screen capture images, in accordance to one embodiment of the present invention. FIG. 23 and FIG. 24 illustrate the initial placement of the ultrasound probe in order to determine the best skin entry point for a perpendicular needle-bone contact. It is important to have a perpendicular approach to the bone; otherwise, the needle may spin down the bone at an angle instead of drilling into the bone. This may result in a painful scraping of the periosteum. In order to avoid this occurrence, the needle is ideally guided perpendicular to the surface of the bone. Therefore, after the correct perpendicular needle trajectory is placed on the screen, the laser disposed onto needle guide 2204 then projects to the skin to mark skin the entry site. This also obviates the need to draw a mark on the skin for an entry site. FIG. 25 illustrates a biopsy needle in correct bone angle contact with the bone surface at the target biopsy site.

In one embodiment of the present invention, the guidance software may include a feature to guide a user to angle ultrasound probe 2206 to an approximately 60 degree angle to the needle, while the user is choosing the trajectory. This will ensure good visualization of the needle. If the needle is too steep compared to the probe, the needle may not be seen. Therefore, this feature of the guidance software may compensate for mistakes in novice user population.

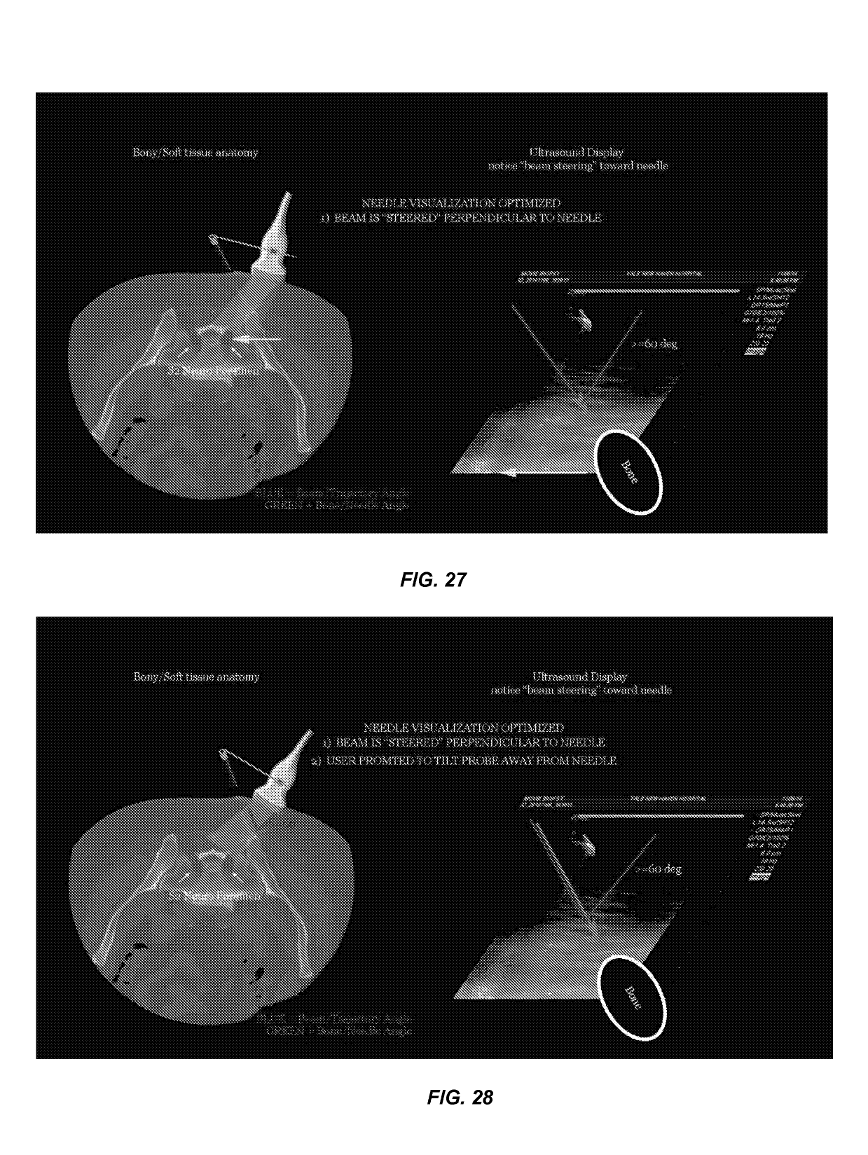

FIG. 26, FIG. 27 and FIG. 28 illustrate ultrasound images for optimal needle visualization achieved through dynamic adjustment of the probe to maintain a perpendicular configuration between the ultrasound probe beam and the biopsy needle, as the needle is advanced towards the biopsy target bone site. In one embodiment of the present invention, the software prompts the user to tilt the probe away from the needle in order to maintain perpendicular beam-needle intersection as the needle is advanced into the tissue towards the bone surface.

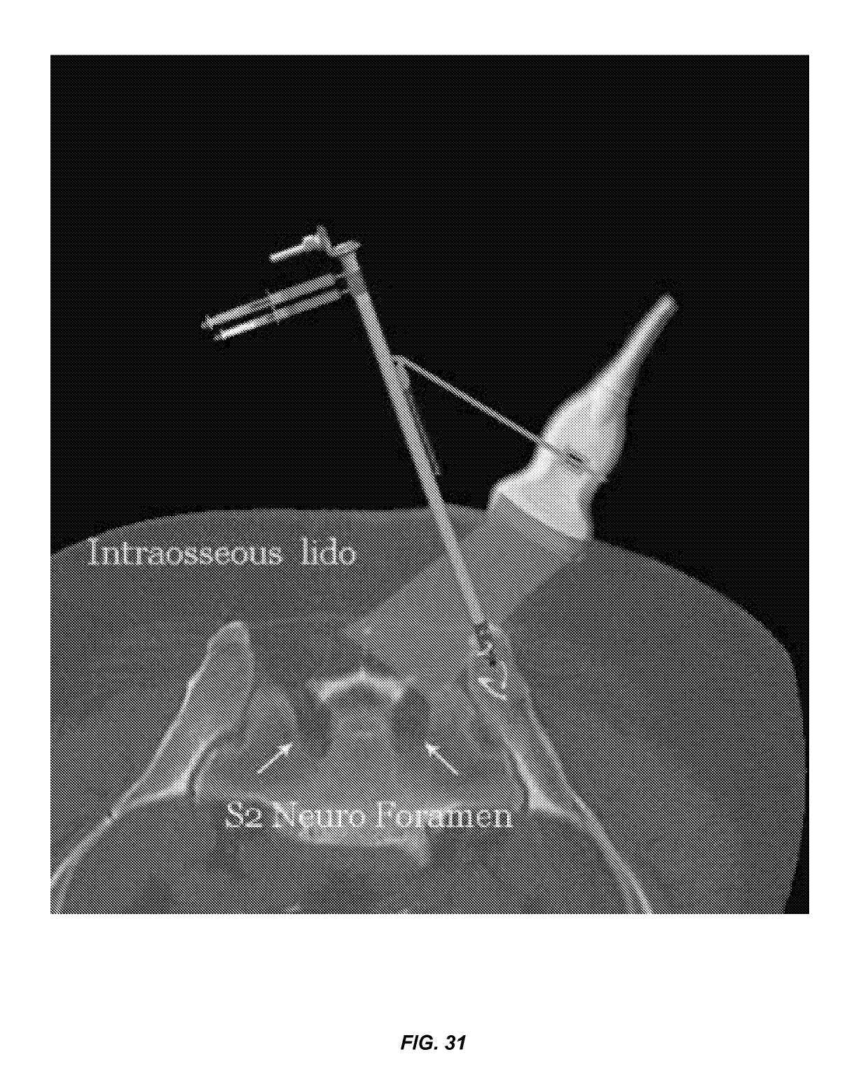

FIG. 28 illustrates a biopsy needle delivering lidocaine into the soft tissue surrounding it for anesthetizing the path of the needle to the bone. Once the bone surface is contacted, lidocaine is delivered by the biopsy needle onto the periosteum to numb the bone drilling site, as illustrated in FIG. 30. Once the biopsy needle has penetrated the cortex, intraosseous lidocaine is delivered into the marrow cavity, as illustrated in FIG. 31, in order to perform a painless aspiration process.

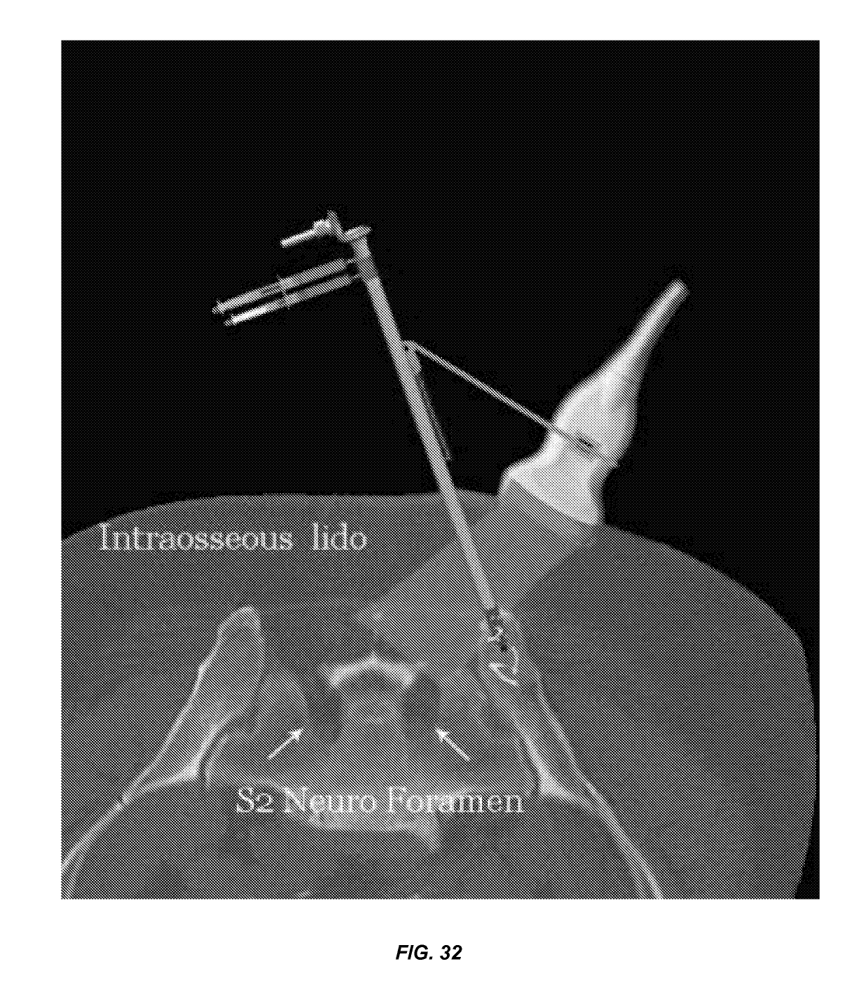

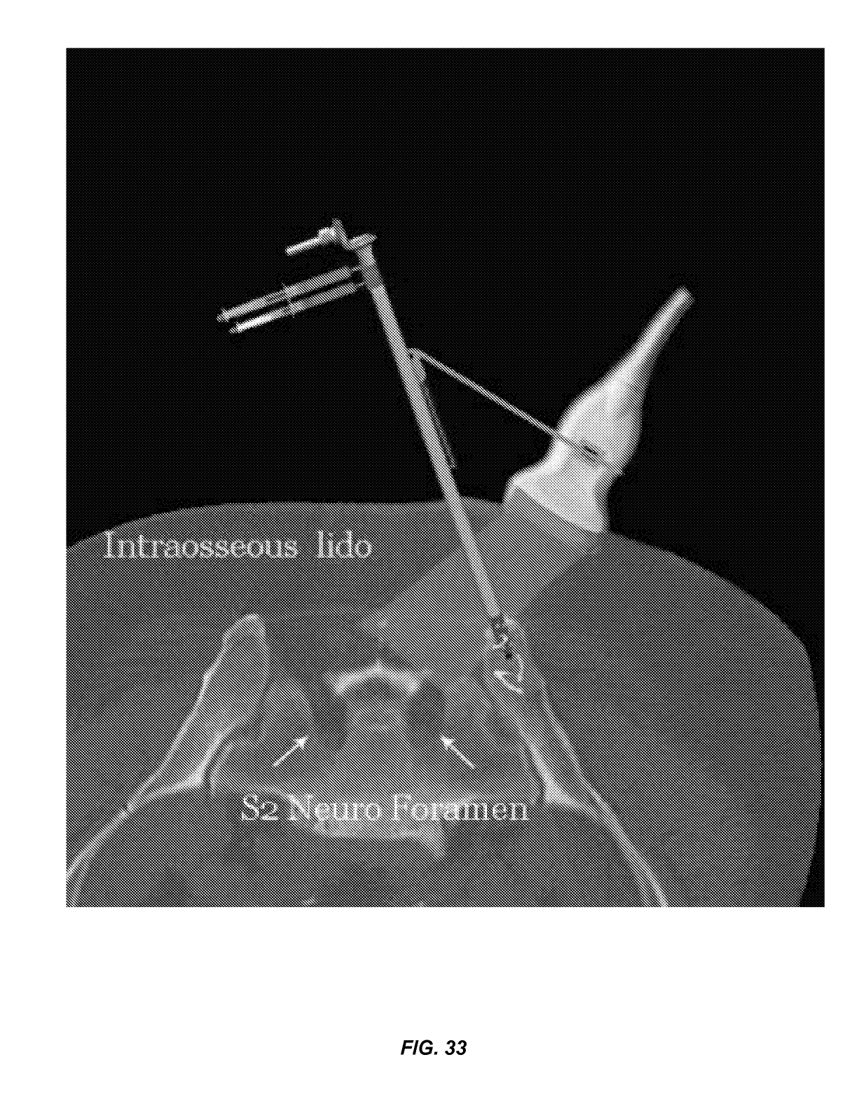

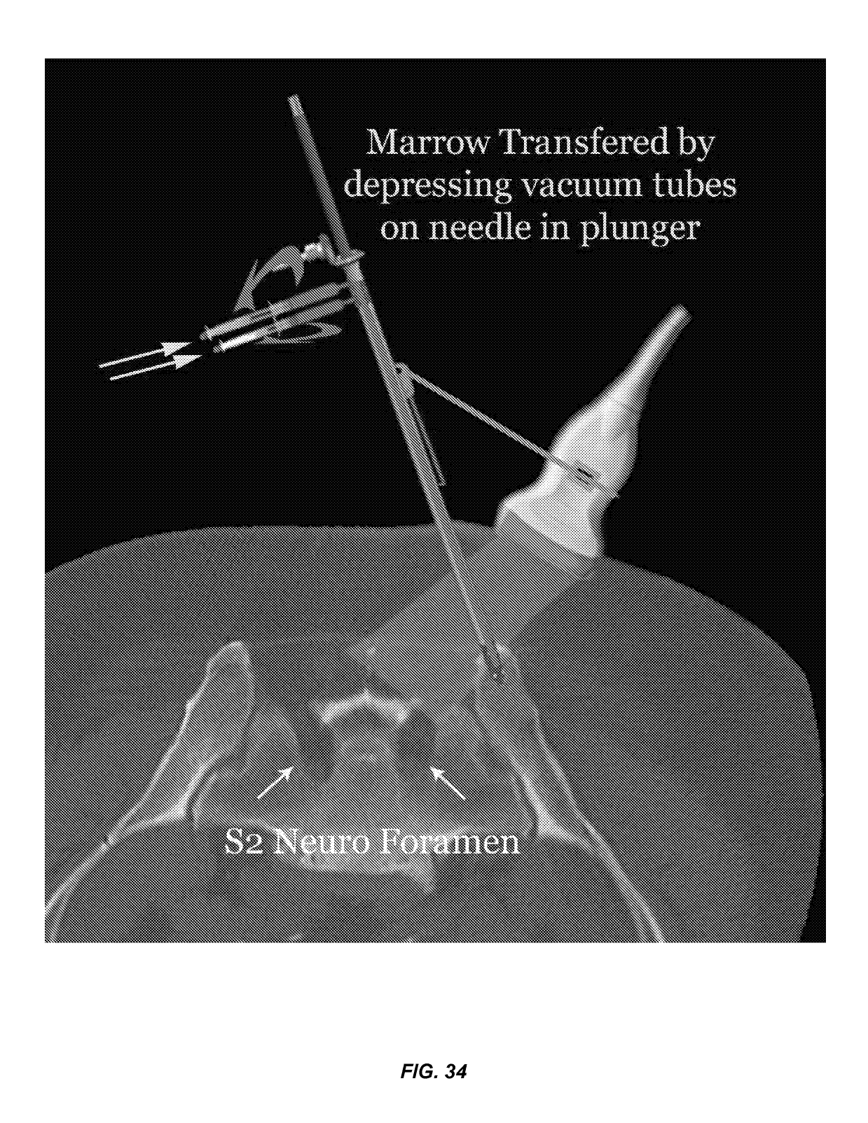

FIG. 32 and FIG. 33 illustrate the process of bone marrow aspiration into the hollow channel provided by the retraction of the central stylet. FIG. 34 illustrates the transfer process of marrow aspirate from the aspiration syringe into sterile vacuum tubes fitted into the plunger of the aspiration syringe. Once aspirate sample has been retrieved, the biopsy needle may be driven further into the marrow cavity space with the central stylet retracted as illustrate in FIG. 35, to collect a solid marrow core sample. The needle may be rotated while being advanced into the marrow cavity space for easier marrow cavity penetration, and also rotated while being withdrawn from the marrow cavity space in order to separate the collected marrow from the surrounding marrow in the marrow cavity.

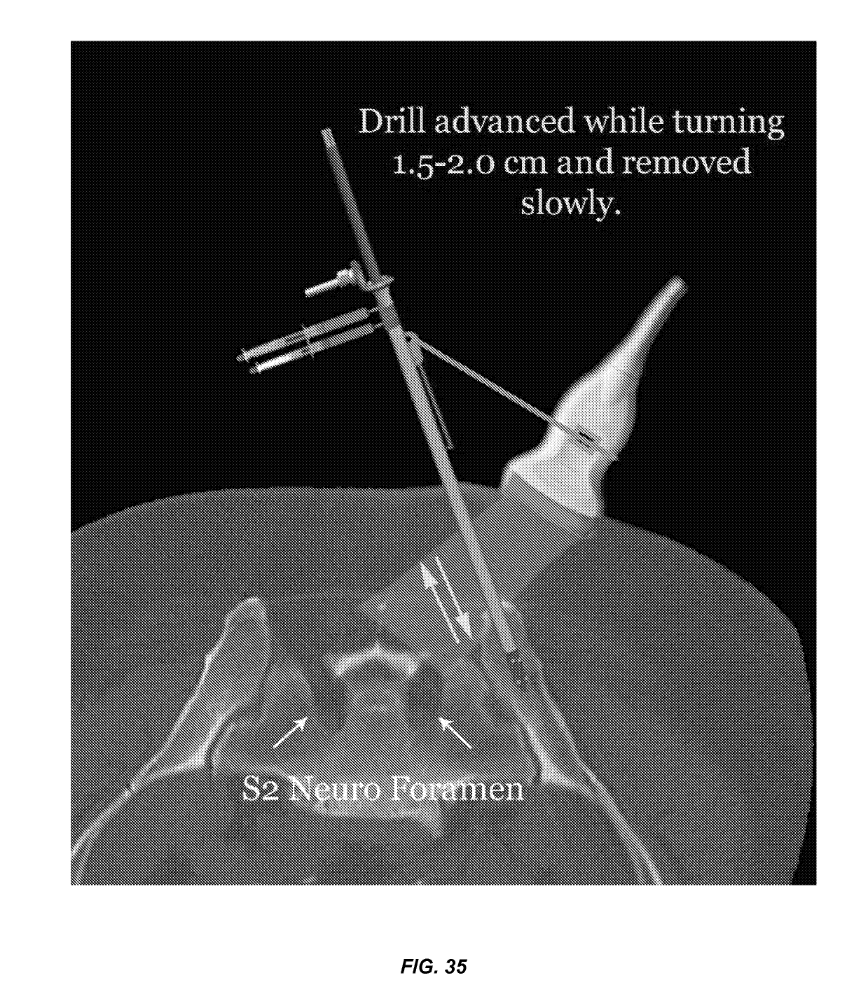

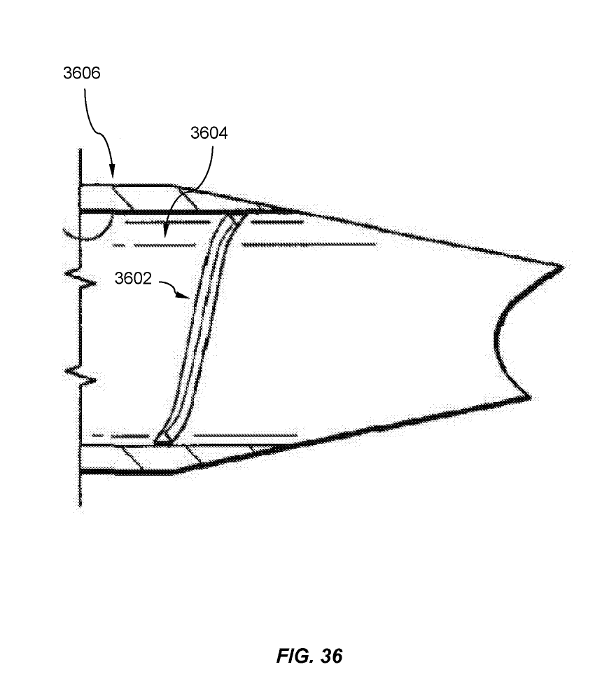

In some embodiments of the present invention, a retaining structure, such as a helical thread, may be incorporated into the design of the biopsy needle as illustrated in FIG. 36. FIG. 36 illustrates a biopsy needle 3600 comprising a helical thread 3602 disposed within a hollow biopsy trough region 3604 at a distal end 3606 of biopsy needle 3600. One purpose of an internal retaining structure, such as helical thread 3602, maybe to ensure that a retrieved sample is retained within biopsy trough region 3604 when biopsy needle 3600 is withdrawn.

In yet another embodiment, it is desirable to provide at least part of the intraosseous administration of lidocaine as an infusion rather than injection, so that lidocaine is administered slowly over several minutes, for example, 4 cc of lidocaine over 2 minutes. Embodiments of the present invention may comprise a mechanism for automatically delivering an infusion at a specific rate. The aforementioned procedure may begin with a small, quick push to break, for example, a small blood vessel and make a channel from the needle hole to the blood vessel, followed by initiating a slow infusion of, for example a therapeutic agent such as an anesthetic agent, from the needle, across the channel, and into the blood vessel. In some cases, it may be necessary to do a slow infusion in order to circumvent the pain experienced by the patient from the increased pressure from the volume of the injection. Slow infusion may also allow time to anesthetize the entire bone.

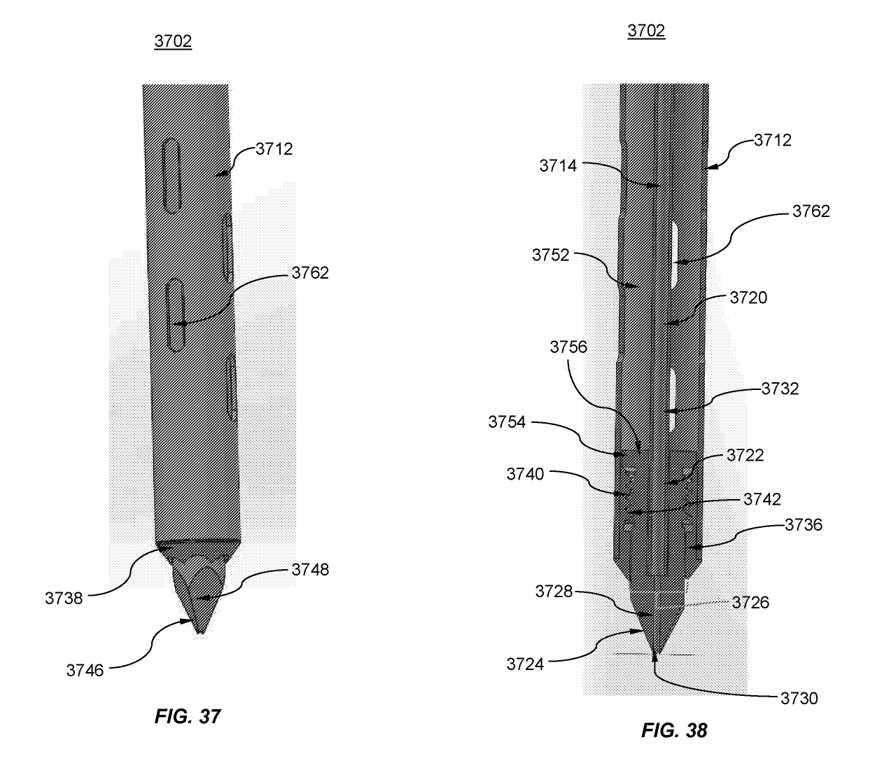

In performing bone marrow biopsies, pain comes from two main sources: (1) pain from drilling into bone and (2) pain from aspirating marrow. With respect to the pain from drilling into bone, as the pain-sensing nerves are on the covering (periosteum) of the cortex of the bone, if anesthetized with lidocaine, the cortical bone can be drilled without any pain. Lidocaine administration is currently done with a separate needle, but lidocaine is not always placed at the site of drilling, therefore, many patients still perceive pain upon bone entry. In one embodiment of the present invention shown in FIGS. 37 and 38, a needle assembly integrates the spinal needle used to anesthetize the periosteum of the bone into a central stylet of an outer sleeve that can be used for obtaining bone core samples. This stylet is solid in other devices and is used to maintain patency of the larger outer needle while entering bone.

The central stylet that has a hollow conduit through which lidocaine can be administered, ensuring lidocaine is administered directly at the site of bone entry. Such a design should reduce or eliminate the pain associated with bone entry. The design of the needle assembly of FIGS. 37 and 38 also eliminate the separate lidocaine step from the procedure.

With respect to the pain from aspirating the marrow, this pain results from pressure changes in the bone and can be excruciating; however, the clinician's underestimate the degree of pain. In an emergency setting, where intraosseous infusions are sometimes performed in lieu of IV infusions, these infusions also cause painful pressure changes in the bone, but are safely and effectively treated using intraosseous lidocaine for whole-bone anesthesia. However, it must be done in a specific way to achieve anesthesia. If lidocaine is administered too quickly, it will itself result in painful pressure change and the medication will flow into the peripheral veins draining the marrow. It must be done as a very slow infusion (about 0.2 ml every 15 seconds) with intermittent pauses to give time for the lidocaine to diffuse into the medullary space. Current bone marrow needles have a volume of ˜1.0-1.5 ml. As a result, when the stylet is removed and a lidocaine syringe placed, an air gap within the needle of that volume, i.e., ˜1.0-1.5 ml, will interpose between the marrow and the lidocaine. Hence, injection would increase bone pressure with air, without anesthetic administration. In the needle assembly of FIGS. 37 and 38, the problem of air gap is removed by providing side openings in the outer sleeve both proximally, where a syringe would attach, and at the distal aspect of the needle that would be in the bone. This design of the needle assembly allows lidocaine to be injected and bone marrow to be aspirated while the stylet remains in place. Therefore no airgap is created with the removal of the stylet.

Also, in current methods of performing a bone marrow biopsy, the bone core is taken from a different bone hole than the hole in which the aspiration of bone marrow is performed, so that the aspiration does not remove marrow cells from the bone core. This is called aspiration artifact. However, this second hole causes increased pain, lengthens the procedure, and increases the degree of scarring/bony sclerosis, as a result of the healing process. Theoretically, this healing process could replace marrow making subsequent sampling less effective, though, this has not been systematically studied. In one embodiment of the present invention illustrated in FIGS. 37 and 38, needle assembly is provided with side openings that allow for aspiration of bone marrow material from side openings in an outer sleeve, while a central stylet remains in place and occludes the end hole. This will preserve the bone in front of the needle, which can subsequently be sampled without aspiration artifact after removal of the central stylet.

FIGS. 37 and 38 show a needle assembly 3702 according to one embodiment of the present invention that includes an outer sleeve 3712 and a stylet 3714. Stylet 3714 includes an upper conduit 3720 that extends into a hollow recess 3722 in a distal end portion 3724 of stylet 3714. A removable distal end portion 3724 extends through and blocks a distal sleeve opening 3726. Upper conduit 3720 is in fluid communication and abuts lower conduit 3728 that extends through distal end portion 3724 and terminates distally at end-opening 3730. Together, upper conduit 3720 and lower conduit 3728 form a stylet conduit 3732. Upper conduit 3720 is larger in diameter than lower conduit 3728. Distal end portion 3724 is mounted in a hollow distal mount 3736 that is mounted in a distal end 3738 of outer sleeve 3712 so that distal end portion 3724 is also mounted in distal end 3738 of outer sleeve 3712. External threads 3740 of distal end portion 3724 engage internal threads 3742 of distal mount 3736 to hold distal end portion 3724 in place in distal mount 3736. Distal end portion 3724 includes a sharp tip 3746 having four faces 3748. Surrounding upper conduit 3720 is a sleeve conduit 3752. Distal end portion 3724 includes an occluding portion 3754 that is disc-shaped that forms an occluded distal end 3756 of sleeve conduit 3752. Outer sleeve 3712 includes side openings 3762 that allow fluids to enter sleeve conduit 3752 from outside outer sleeve 3712. As shown in FIGS. 37 and 38, stylet conduit 3732 is not in fluid communication with sleeve conduit 3752.

The sharp tip of the stylet of the needle assembly shown in FIGS. 37 and 38 allows the needle assembly to be drilled into the marrow of a bone. In the needle assembly shown in FIGS. 37 and 38, a liquid anesthetic, such as lidocaine, may be pumped through the conduit of the stylet and administered directly at the site of bone entry through the distal end opening of the stylet. The side openings needle assembly shown in FIGS. 37 and 38 allow for aspiration of bone marrow material through the side openings of the outer sleeve. Because the stylet conduit is not in fluid communication with the sleeve conduit, the liquid anesthetic can be administered through the distal opening of the stylet at the same time as aspiration is being performed through the side openings. Furthermore, because bone marrow cells are only aspirated through the side openings while the stylet remains in place, the bone in front of the of the distal end portion of the stylet is preserved. This allows the bone core in front of the distal end of the stylet to be subsequently sampled by removing the stylet. The stylet can be removed by unscrewing the distal end portion of the stylet from the distal mount.

In order to maintain a similar resistance to fluid flow within the aspiration needle as compared to current devices, while leaving the central stylet in place, the stylet is made with a smaller diameter, as shown in FIGS. 37 and 38. This puts the stylet at risk for breaking due to torque of drilling into bone. The outer sleeve of the needle assembly is therefore “drilled” opposite of the external threads of the distal end portion of the stylet, thereby, ensuring a transfer of the forces from the stylet to the outer sleeve via the external threads of the distal end portion of the stylet that engage the internal threads of the distal mount of the outer sleeve.

The distal end portion of the stylet completely occludes the distal opening of the outer sleeve so that no aspiration or lidocaine infusion is performed out of the opening of the outer sleeve. This preserves the bone core specimen during the aspiration, allowing both the bone core and the aspiration performed in a single hole rather than two holes.

After the aspiration is performed removing liquid bone marrow, the inner stylet removed; the empty outer sleeve may be advanced into the bone by a distance such as 2 cm (World Health Organization suggests specimen length be at least 1.5 cm). Maintaining this core specimen within the needle as the needle is withdrawn is an important part of the procedure, and the internal threads of the distal mount of the outer sleeve serve as a “barb” to maintain the core specimen within the needle assembly.

With respect to the needle assembly of FIGS. 37 and 38, the use of multiple side openings in the needle assembly for aspiration, instead of a single end-opening to increase the area of marrow being aspirated, may also reduce hemodilution using the needle assembly to perform a bone marrow biopsy. In addition, the needle assembly of FIGS. 37 and 38 may mitigate any hemodilution that a lidocaine infusion, such as a 2 ml of lidocaine infusion, may induce prior to aspiration, by spreading the lidocaine over a wider area. This may also increase rate of onset of anesthesia for aspiration. The needle assembly may also only need to be advanced minimally after the infusion to sample a “new” pocket of marrow.

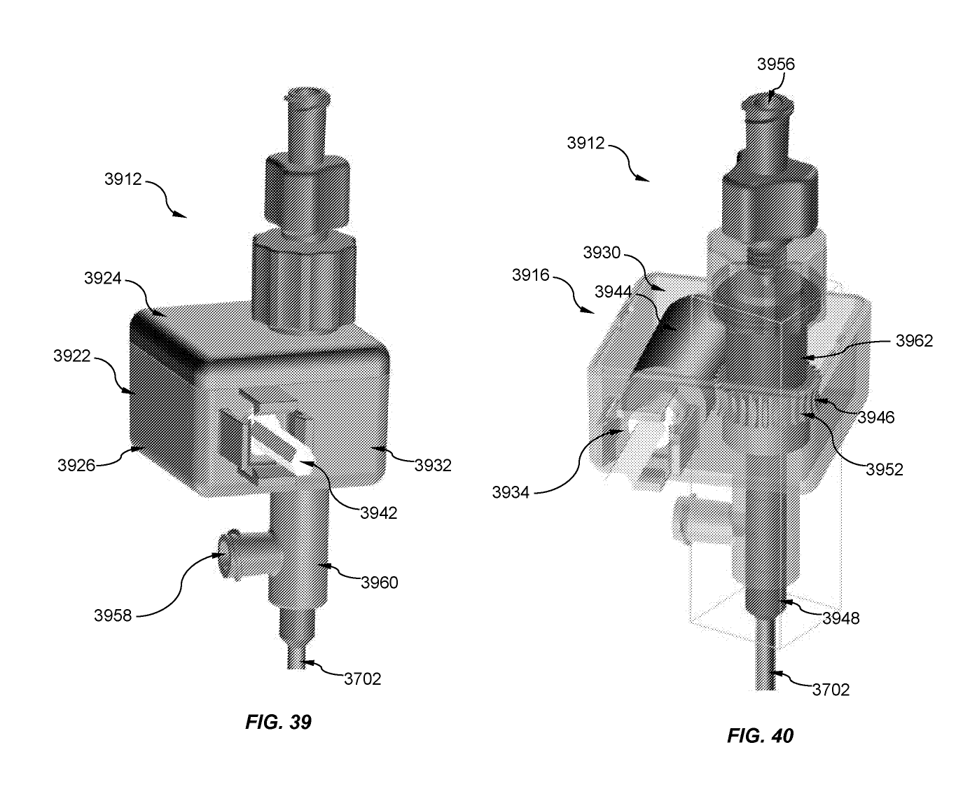

FIGS. 39, 40, 41, 42, 43, 44 and 45 shows a right angle gear mechanism that may be used with a drilling device according to one embodiment of the present invention. In the right angle gear mechanism of FIGS. 39, 40, 41, 42, 43, 44 and 45, the drive shaft of the gear mechanism is at right angle to the axis of rotation of the needle assembly driven by the right angle gear mechanism.

FIGS. 39, 40, 41, 42, 43, 44 and 45 show a drill device 3912 that drive needle assembly 3702 according to one embodiment of the present invention. Drill device 3912 includes gear mechanism 3916. Gear mechanism 3916 includes a gearbox 3920 having a gearbox casing 3922. Gearbox casing 3922 having an upper casing portion 3924 and a lower casing portion 3926. Removing upper casing portion 3924 from lower casing portion 3926 allows access to various parts of gearbox 3920 inside gearbox casing 3922. A side 3932 of gearbox casing 3922 includes an access port 3934 through which extends a hexagonal cylindrical drill shaft 3942 that drives a worm gear 3944, that in turn, drives a needle assembly holder gear 3946 mounted on a rotatable needle assembly holder 3948 holding needle assembly 3702 to thereby rotate rotatable needle assembly holder 3948 and needle assembly 3702. Teeth (not shown) of worm gear 3944 engage teeth 3952 of needle assembly holder gear 3946. Drill device 3912 includes a luer lock access port, i.e., proximal port 3956, that allows a container containing a liquid anesthetic (not shown), such as a syringe, may be connected to drill device 3912 so that the container is in fluid communication with stylet conduit 3732. Drill device 3912 also includes a luer lock access port, i.e., side port 3958 of a distal cylindrical portion 3960 of lower casing portion 3926 of gearbox casing 3922, that allows a suction generator (not shown), such as a syringe, to be connected to drill device 3912, so that the suction generator is in fluid communication with sleeve conduit 3752 for aspirating bone marrow material through side openings 3762. Drill device 3912 includes sealable port 3962 having a barrel-shaped lower portion 3964 and an upper portion 3966. Upper portion 3966 includes an external screw thread 3968 and a wide-mouthed opening 3970. Drill device 3912 includes a septum seal 3972, i.e., a washer-shaped piece of deformable material such as silicone, rubber, etc. and a septum seal cap 3974 having internal screw thread 3976.

Accessing the needle assembly from the side, while the stylet remains in place, is accomplished by two features of the drill device of FIGS. 39, 40, 41, 42, 43, 44 and 45: (1) an air-tight seal around the inner stylet; an air-leak would preclude the vacuum required to extract bone marrow from the bone, and (2) a side opening in the needle assembly connected to a luer lock access port.

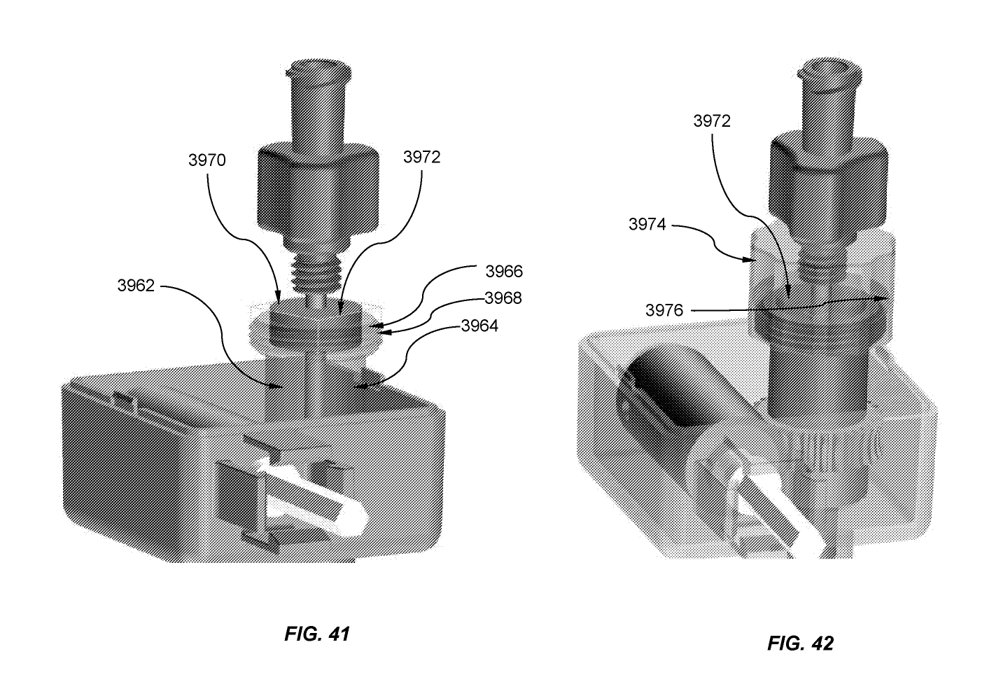

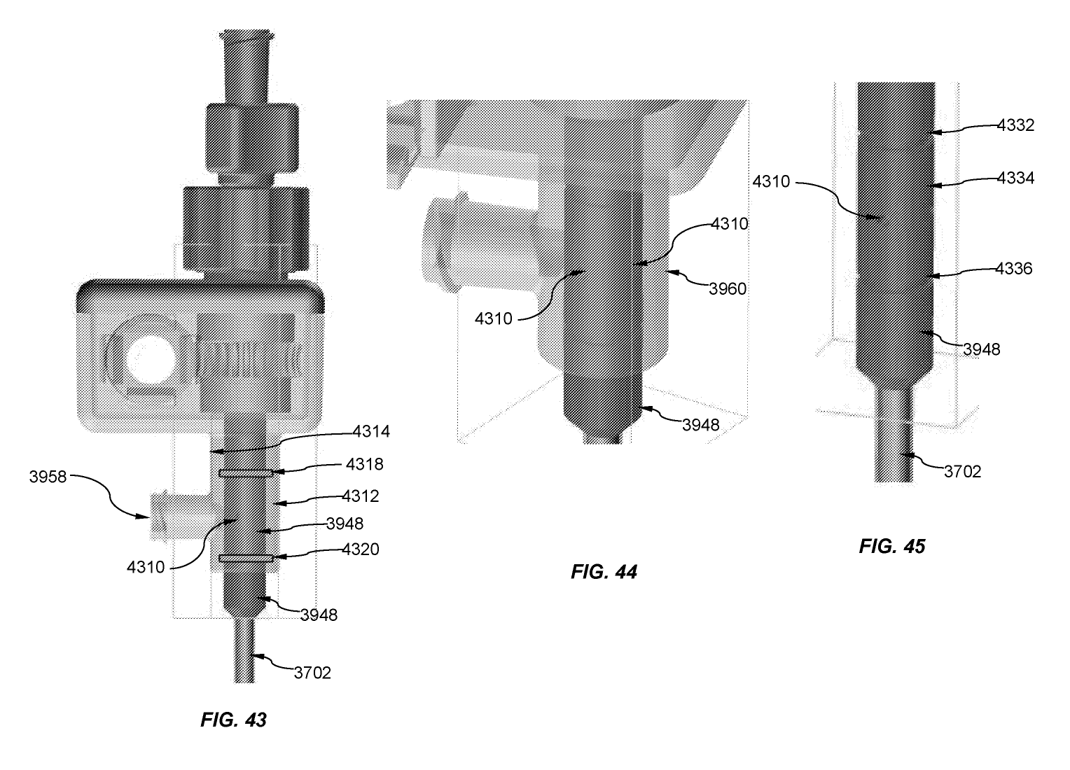

FIG. 41 shows septum seal 3972 in place in wide-mouth opening 3970. FIG. 42 shows septum seal cap 3974 being screwed onto wide-mouthed opening 3970 by internal thread of septum seal cap 3974 being screwed onto external screw thread 3968 of upper portion 3966 so that septum seal 3972 is compressed into wide-mouth opening 3970 to thereby form an air-tight seal around needle assembly 3702. This air-tight seal allows suction to effectively be applied to sleeve conduit 3752 through side port 3958 that is in fluid communication with side openings 3762 of sleeve conduit 3752 through holder side ports 4310 in needle assembly holder 3948, shown in FIGS. 43, 44 and 45. This air-tight seal also allows the aspiration to occur no matter the rotational position of the needle., due to the circumferential connecting chamber around needle assembly holder 3948.

Although multiple sleeve side ports are shown in FIGS. 43, 44 and 45, in some embodiments of the present invention, there may only be a single sleeve side port.

A coupling mechanism is used to allow a stationary syringe communicate with the lumen of the rotating needle assembly. This is done by having the syringe connect via lure lock to an air-tight fluid-tight chamber around the needle assembly or a needle assembly holder holding the needle. This chamber is a space circumferentially around the needle assembly or needle assembly holder. Thus, the fluid can flow to and from the needle and syringe via the chamber no matter where the openings in the needle are positioned as the needle turns, and can even administer fluid or aspirate while the drill is spinning.

FIGS. 43, 44, and 45 show such an air-tight, fluid-tight chamber, i.e., chamber 4312, according to one embodiment of the present invention, around holder side ports 4310. Chamber 4312 is formed by interior wall 4314, i.e., an inner surface, of distal cylindrical portion 3960 of lower casing portion 3926 of gearbox casing 3922, proximal O-ring 4318 and distal O-ring 4320 around needle assembly holder 3948. Proximal O-ring 4318 fits into a circumferential groove 4332 on an exterior surface 4334 of needle assembly holder 3948. Distal O-ring 4320 fits into circumferential groove 4336 on an exterior surface 4340 of needle assembly holder 3948.

The O-rings may be made from a deformable material such as silicone, rubber, etc.

The use of worm gear in the drill device of FIGS. 39, 40, 41, 42, 43, 44 and 45 allows both a reduction of speed and increased torque, but also access to the needle assembly without having to remove the drill. This increases the efficiency of the procedure.

Although in the drill device of FIGS. 39, 40, 41, 42, 43, 44 and 45, the side luer lock access port is only shown providing access to the sleeve conduit. In other embodiments of the present invention, the side luer lock access port could prove access to both the sleeve conduit and stylet conduit.

Although in FIGS. 39 and 40, the right angle gear mechanism is shown as being at least partially contained in the drill casing; however, the right angle gear mechanism may be entirely contained in the drill casing or completely external to a gearbox casing.

A drill device of the present invention may or may not have an integrated medication delivery device, such as a pump, the pump is a peristaltic pump, a piezoelectric pump or any other type of pump. This will allow the user to administer fluid, such as lidocaine, at the touch of the button. An example of such use would be to administer lidocaine into the soft tissues as the needle is advanced from skin to bone, without having to detach the drill handle from the needle, and with only a single hand. This will free the other hand to hold an ultrasound probe or palpate the bony landmarks. A control board integrates the control for both the drill motor and the pump. The pump can administer fluid at different rates, for example, a higher rate for subcutaneous tissues, and a lower rate for infusion into the bone. The importance of having a pump to perform the infusion into the bone is to decrease user error. For adequate whole-bone anesthesia, lidocaine should be infused at a specific low rate, such as 2 ml over 4 minutes, with intermittent pauses to allow lidocaine to diffuse throughout the venous channels. If the user injects lidocaine into the bone too quickly, it will simply drain into the venous system and increase pressure (causing pain) without creating bony anesthesia.

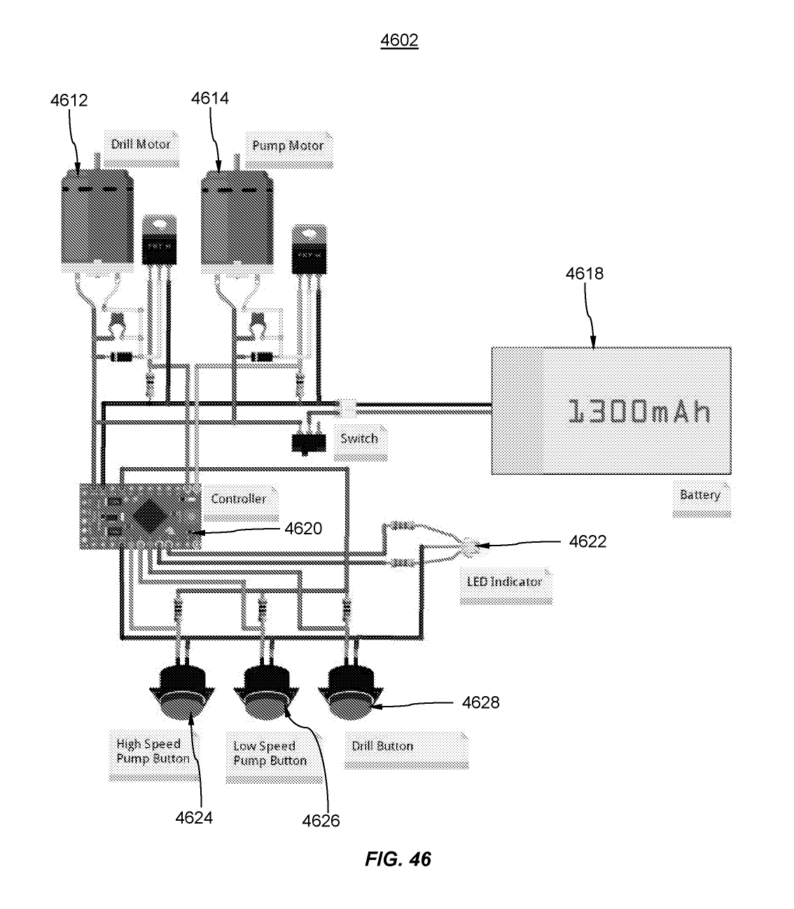

FIG. 46 shows a combination control apparatus 4602, a drill that includes both drilling and pumping functions, according to one embodiment of the present invention. Control apparatus 4602 includes a drill motor 4612 that drives the drill, a pump motor 4614 that drives the pump, a battery 4616 that supplies power to the apparatus, a switch 4618 for turning the drill on, a controller 4620 that controls the overall operation of the drill, a LED indicator 4622 that comprises various LED lights that indicate to a user the operation(s) currently being performed by the drill, a high speed pump button 4624 that when pressed by a user causes the pump to operate at a high speed, a low speed pump button 4626 that when pressed by a user causes the pump to operate at low speed, and a drill button 4628 that when pressed by a user causes the drill of the drill device to operate.

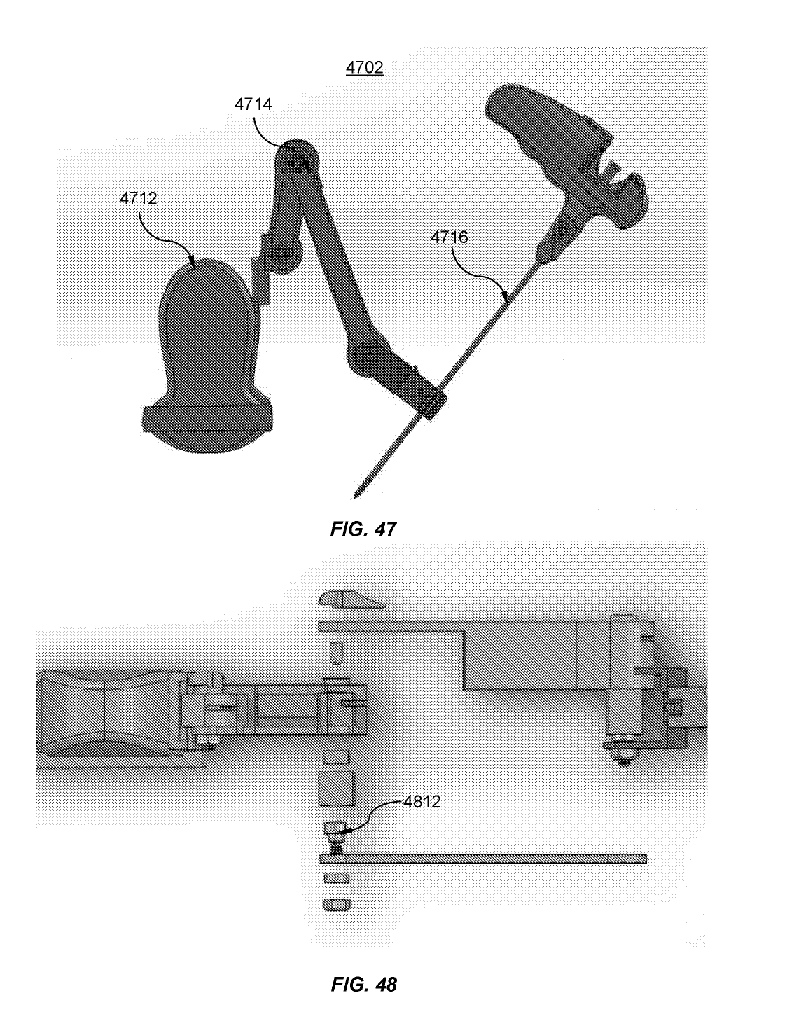

FIGS. 47 and 48 show an ultrasound guidance device 4702, according to one embodiment of the present invention, including an ultrasound probe 4712, an articulated guidance arm 4714, which attaches to ultrasound probe 4712, and a needle assembly 4716 in a needle assembly holder 4718. FIG. 48 shows a potentiometer 4812 that allows the software to compute the relative angle of needle assembly 4716 to ultrasound probe 4712. Ultrasound guidance device 4702 may be used to control the movement of needle assembly 4716.

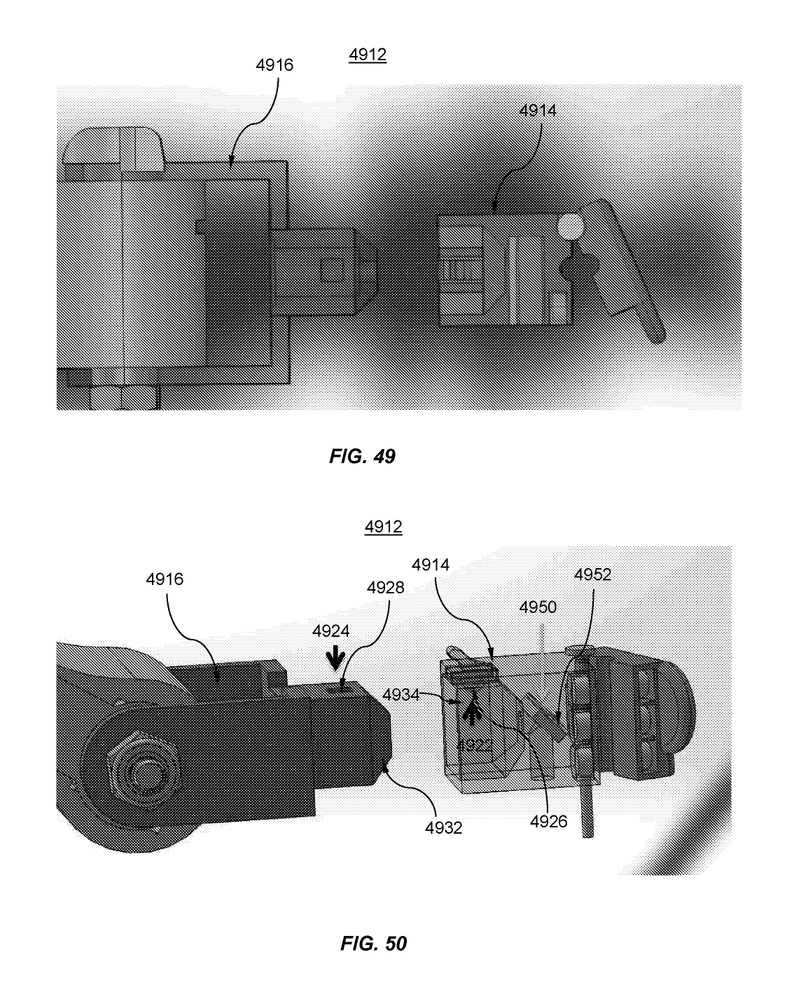

FIGS. 49 and 50 show an interface 4912 between a sterile disposable component 4914 and a non-sterile reusable component 4916 of needle assembly holder 4718. A method of attachment is noted by arrows 4922 and 4924 that show a releasable tab 4926 that fits into a recess 4928 when projecting end 4932 of non-sterile reusable component 4916 is inserted into recess 4934 of sterile disposable component 4914. A laser embodied within the red articulating component shines a light, indicated by arrow 4950, onto a mirror 4952 within sterile disposable component 4914. When the appropriate angle has been chosen by the user, the laser turns on and shines adjacent to where the user should administer local anesthesia and perform the dermatotomy. This obviates the need to use indelible ink and saves time during the procedure.

In one embodiment of the present invention, the ultrasound guidance devices provide real-time feedback to the user as he/she plans the trajectory to the target, as to the appropriate angle between the planned trajectory of the needle. This is done prior to the insertion of the needle assembly. The plan is done with the needle guide, and therefore, the needle assembly will advance exactly as planned.

### EXAMPLES

A 10-patient preliminary in-vitro trial of the effect of lidocaine on bone marrow is conducted where lidocaine was mixed in-vitro to human bone marrow aspirates to evaluate its effect, if any, on the ability to culture the cells and the bone marrow smear. Cell culture demonstrated no significant difference in mitotic rate between the control and lidocaine groups. This suggests that lidocaine will not affect the ability of cells to be cultured for further analysis as in cytogenetics. Bone marrow smears were performed, but have not yet been analyzed.

All documents, patents, journal articles and other materials cited in the present application are incorporated herein by reference.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A device comprising:
a needle assembly comprising:
a hollow tubular sleeve having a hollow opening therethrough,
a stylet mounted in the hollow opening of the hollow tubular sleeve and having a first conduit therethrough, and
one or more side openings,
a liquid pressure generator in fluid communication with the first conduit and configured to apply pressure to liquid in the first conduit,
wherein when a container containing a liquid anesthetic agent is in fluid communication with the first conduit and the liquid pressure generator applies pressure to the liquid anesthetic agent, the liquid anesthetic agent is forced through the first conduit and out through one or more end-openings of the stylet, and
wherein when the device is inserted in bone marrow so that side openings are exposed to the bone marrow and a suction generator applies suction to a second conduit extending at least partway through the needle assembly, at least a portion of the bone marrow is withdrawn into the device through the side openings.

2. The device of claim 1, wherein the first conduit and second conduit are different conduits.

3. The device of claim 1, wherein the stylet includes a removable distal end portion that occludes the second conduit and wherein the first conduit is configured to aspirate a bone core sample when the removable distal end portion is removed.

4. The device of claim 1, wherein the side openings are part of the hollow tubular sleeve.

5. The device of claim 1, wherein the container, liquid pressure generator and suction generator are one or more syringes.

6. The device of claim 1, wherein the liquid anesthetic agent is lidocaine.

7. The device of claim 1, wherein the device comprises:
a needle assembly holder that holds the needle assembly and includes a needle assembly holder gear, and
a worm gear configured to drive the needle assembly holder gear to thereby rotate the needle assembly holder and the needle assembly.

8. The device of claim 1, wherein the device comprises:
a casing having a distal cylindrical portion,
a needle assembly holder having one or more holder side ports, and
a chamber formed by an inner surface of the distal cylindrical portion, a proximal O-ring sealing a first space between the casing and the needle assembly holder and a distal O-ring sealing a second space between the casing and the needle assembly holder,
wherein the needle assembly holder is rotatable,
wherein the needle assembly holder is mounted to the casing through a portion of a casing that surrounds and is spaced from the needle assembly holder,
wherein the needle assembly is mounted in the needle assembly holder,
wherein the casing includes a port in fluid communication with the one or more holder side ports of the needle assembly holder, and
wherein the chamber surrounds the one or more holder side ports.

9. The device of claim 1, wherein the device comprises a ultrasound guidance device comprising an ultrasound probe, an articulated guidance arm attached to the ultrasound probe, and a needle assembly holder mounted on the articulated guidance arm, and wherein the needle assembly is held by the needle assembly holder.

10. A method comprising administering a liquid anesthetic to a site of drilling in a bone through an opening in a tip of a needle assembly drilling into the bone as the needle assembly drills into the bone at the site of drilling.

11. The method of claim 10, wherein the method comprises aspirating bone marrow from the bone through the needle assembly without removing the needle assembly from the site of drilling.

12. The method of claim 11, wherein the method is conducted by a device comprising:
a needle assembly comprising:
a hollow tubular sleeve having a hollow opening therethrough,
a stylet mounted in the hollow opening of the hollow tubular sleeve and having a first conduit therethrough, and
one or more side openings
a liquid pressure generator in fluid communication with the first conduit and configured to apply pressure to liquid in the first conduit, and
wherein when a container containing a liquid anesthetic agent is in fluid communication with the first conduit and the liquid pressure generator applies pressure to the liquid anesthetic agent, the liquid anesthetic agent is forced through the first conduit and out through one or more end-openings of the stylet, and
wherein when the device is inserted in bone marrow so that side openings are exposed to the bone marrow and a suction generator applies suction to a second conduit extending at least partway through the needle assembly, at least a portion of the bone marrow is withdrawn into device through the side openings.

13. The method of claim 12, wherein the first conduit and second conduit are different conduits.

14. The method of claim 12, wherein the stylet includes a removable distal end portion that occludes the second conduit and wherein the first conduit is configured to aspirate a bone core sample when the removable distal end portion is removed.

15. The method of claim 12, wherein the side openings are part of the hollow tubular sleeve.

16. The method of claim 12, wherein the container, liquid pressure generator and suction generator are one or more syringes.

17. The method of claim 12, wherein the liquid anesthetic agent is lidocaine.

18. The method of claim 12, wherein the device comprises:
a needle assembly holder that holds the needle assembly and includes a needle assembly holder gear, and
a worm gear configured to drive the needle assembly holder gear to thereby rotate the needle assembly holder and the needle assembly.

19. The method of claim 12, wherein the device comprises:
a casing having a distal cylindrical portion,
a needle assembly holder having one or more holder side ports, and
a chamber formed by an inner surface of the distal cylindrical portion, a proximal O-ring sealing a first space between the casing and the needle assembly holder and a distal O-ring sealing a second space between the casing and the needle assembly holder,
wherein the needle assembly holder is rotatable,
wherein the needle assembly holder is mounted to the casing through a portion of a casing that surrounds and is spaced from the needle assembly holder,
wherein the needle assembly is mounted in the needle assembly holder,
wherein the casing includes a port in fluid communication with the one or more holder side ports of the needle assembly holder, and
wherein the chamber surrounds the one or more holder side ports.

20. The method of claim 12, wherein movement of the needle assembly is controlled by an ultrasound guidance system.
